(19) United States
(12) Patent Application Publication
(10) Pub. No.: US 2020/0095055 A1
(43) Pub. Date: Mar. 26, 2020

(54) METHOD AND APPARATUS FOR INDIRECTLY DETERMINING THE DISPENSING OF MEDICATION FROM A CONTAINER

(71) Applicant: Kali Care, Inc., Mountain View, CA (US)

(72) Inventors: Philippe Cailloux, Sunnyvale, CA (US); Abhijit Kalamkar, Sunnyvale, CA (US); Navid Nick Afsarifard, Atherton, CA (US); Seungju Baek, Santa Clara, CA (US)

(21) Appl. No.: 16/137,369

(22) Filed: Sep. 20, 2018

(51) Int. Cl. B65D 83/38; A61F 9/00

(52) U.S. Cl. CPC B65D 83/38; B65D 47/18; A61F 9/0008

(57) ABSTRACT

A body is configured to accept a medication container in a container cavity defined therein. The body may approximate a half-cylinder with the container disposed in the concavity, and held in place with flexure tabs or other restraints. A force sensor is disposed on the interior of the body in communication with the container cavity and container. Dispensing force applied to the container from the body side do not reach the container to dispense medication; dispensing force applied from the open side do, and also cause the container to press against the force sensor. This indirect, transmitted force is detected with the force sensor, and the force data is evaluated in a processor. In response to the force data corresponding with dispensing medication, the processor registers a medication event, such as by storing the event, displaying the event, or communicating the event to a third party.

### FIELD OF THE INVENTION

Various embodiments concern technologies for acquiring information indicating the dispensing of medication or other substances. More particularly, various embodiments relate to technologies for determining whether medication is being dispensed, prepared for use, etc., through indirectly detecting pressure, contact, etc., applied to the container.

### BACKGROUND

A substantial portion of medications are not taken as prescribed. By some estimates, in clinical practice up to 50% or more of medications either may not be taken at all or may be taken with significant deviations from what is prescribed for the patient. For example, doses of a medication may be skipped, the medication may not be taken at the right intervals, at the right times, in the right dose, applied in the correct manner, etc. Such deviation from a prescribed medication regimen may be referred to broadly as “nonadherence.” Nonadherence to prescribed medication regimens may have dramatic negative effects on health and/or healthcare costs, whether considering individuals or societies collectively.

Nonadherence may be even more common in clinical research, wherein some estimates indicate nonadherence of up to 70% or more. Nonadherence in a research context also presents other potential concerns. For example, testing of new medications typically may include efforts to determine the effectiveness of the medication, what side effects occur, how severe those side effects may be, in what fraction of the population those side effects occur, etc. Thus, nonadherence in a research setting may distort the basic understanding of a medication, e.g., if a medication is in fact highly effective if taken as prescribed but ineffective or dangerous if not taken properly, poor adherence within a clinical trial may result in data showing that the medication is not effective (when the actual problem is that it was not taken correctly).

One matter complicating issues related to nonadherence is that reliable data on the existence, degree, and form(s) of nonadherence present may be difficult to acquire. Whether for an individual, a larger population, or even a carefully selected and/or monitored group such as the subjects in a clinical trial, authentic data on how much nonadherence is taking place, among whom, and in what forms (e.g., missing doses, taking the medication incorrectly, etc.) may not be available through conventional sources. Without such authenticated data it may not even be known how much nonadherence is taking place (beyond estimates), much less what the specific impacts of nonadherence may be in a given case.

At least in principle, it may be possible to detect, record, and/or report the use of a medication through making medication containers “smart,” in such way that the containers themselves may detect that medication has been dispensed and/or used. For example, sensors may be disposed on or incorporated into a container. However, such an approach also presents challenges, such as potential fragility of such sensors or the possibility that events other than dispensing medication may result in spurious data being generated by the sensors. For example, medication containers that are handled and/or carried regularly may be subject to various environmental hazards, e.g., the container may be dropped, sat upon (for example if kept in a pocket), bumped or scraped by other objects in a pocket or bag, etc., possibly resulting in damage. Similarly, sensors may be triggered by similar conditions, for example a force sensor on a container kept in a pocket may record forces applied to the container by other objects in the pocket, etc., possibly causing data to be generated that may incorrectly suggest that someone squeezed the container in order to dispense medication.

Also, medication containers frequently may be disposable. For example, a container may hold a 30-day supply of medication, a single dose, etc., with the expectation that a user may dispose of the container when the medication has been consumed (or has expired, etc.). If such a medication container includes sensors that are in and/or on the container, then the sensors (and/or other electronics or elements) may be disposed of with the containers. This may present issues with regard to manufacturing a smart container at a suitable cost as to be disposable, and/or dealing with the presence of sensors etc. within recycling or waste streams. On the other hand, while reusing a medication container may be possible in principle to avoid such cost/disposal concerns, reuse may itself raise issues with regard to sterility and safety, regulatory approval, and so forth.

### SUMMARY

This disclosure contemplates a variety of systems, apparatus, methods, and paradigms for determining indirectly whether medication has been dispensed from a container.

In one embodiment an apparatus is provided that includes a structural body (or simply “a body”), the body defining a container cavity adapted to accommodate a medication container so as to move with the body, a force aperture adapted to pass dispensing force to the container and to receive the medication container into the container cavity in an inward lateral motion, and a dispensing aperture adapted to enable the neck of the medication container to extend therethrough to enable the container to dispense medication. The body includes flexible arms adapted to releasably restrain the container within the container cavity, a back obstacle adapted to obstruct outward lateral motion of the container from the container cavity other than via the force aperture, an upper obstacle adapted to obstruct an upward vertical motion of the container from the container cavity (the dispensing aperture being defined therein), and a lower obstacle adapted to obstruct a downward vertical motion of the container from the container cavity.

The apparatus includes a spine engaged with the body, the spine defining a spine component cavity therein such that when the spine is engaged with the body the container cavity is between the force aperture and the spine component cavity, and a foot engaged with the body, the foot defining a foot component cavity. The apparatus also includes a proximity sensor disposed within the spine component cavity and adapted to generate proximity data in response to a proximity of a finger thereto from outside the body, a disposition sensor disposed within the foot component cavity and adapted to generate disposition data including position data, translation data, orientation data, and/or rotation data in response to the disposition of the body, and a force sensor disposed within the spine component cavity and adapted to generate force data in response to a transmitted force applied thereto from the medication container.

The apparatus further includes a processor disposed within the body and in communication with the force sensor, the proximity sensor, and the disposition sensor, a data store in communication with the processor, an outputter in communication with the processor, and a communicator in communication with the processor. The processor is adapted to determine whether the proximity data from the proximity sensor corresponds with a dispensing proximity of the finger to the proximity sensor associated with dispensing the medication from the container, and in response to the proximity data corresponding with the dispensing proximity, activate the disposition sensor and the force sensor. The processor is also adapted to determine whether the disposition data from the disposition sensor corresponds with a dispensing disposition of the body with the container therein associated with dispensing the medication from the container, and determine whether the force data from the force sensor corresponds with a dispensing force being applied to the medication container and the transmitted force being applied to the force sensor by the medication container in response to the dispensing force. The processor is further adapted to register a medication event in response to the disposition data corresponding with the dispensing disposition and the force data corresponding with the dispensing force.

The body, the force aperture, and the force sensor are configured such that the transmitted force is generated in response to the dispensing force being applied to the medication container through the force aperture, and such that the transmitted force is not generated in response to the dispensing force being applied to the medication container other than through the force aperture.

Registering the medication event includes storing the medication event and a medication event time thereof in the data store, outputting the medication event and the medication event time via the outputter, and communicating the medication event and the medication event time to an external entity via the communicator.

In another embodiment an apparatus is provided that includes a body, the body defining a container cavity adapted to accommodate a medication container such that the medication container moves with the body, and a force aperture adapted to pass a dispensing force to the container, the force aperture further being adapted to receive the medication container into the container cavity. The body includes a restraint adapted to releasably restrain the medication container in the container cavity, a force sensor adapted to generate force data in response to a transmitted force applied thereto from the medication container, and a processor in communication with the force sensor. The processor is adapted to determine whether the force data from the force sensor corresponds with dispensing a medication from the medication container, and in response to the force data corresponding with dispensing the medication, register a medication event. The body, the force aperture, and the force sensor are configured such that the transmitted force is generated in response to the dispensing force being applied to the medication container through the force aperture, and such that the transmitted force is not generated in response to the dispensing force being applied to the medication container other than through the force aperture. Registering the medication event includes storing the medication event and a medication event time thereof, outputting the medication event and the medication event time, and communicating the medication event and the medication event time to an external entity.

The restraints may include a flexible arm adapted to releasably restrain the container within the container cavity via mechanical interference therewith, an adhesive strip adapted to restrain the container within the container cavity via adhesion thereto, a two-part hook-and-loop band adapted to restrain the container within the container cavity via hook-and-loop engagement of the band around the container, an elastic band adapted to restrain the container within the container cavity via the elastic band being disposed around the container, a tie cord adapted to restrain the container within the container cavity via the tie cord being tied around the container, and a magnet adapted to restrain the container within the container cavity via magnetic engagement between the magnet and the container.

The body may include a shell forming at least half of a circumference of a cylinder, the container cavity being defined as a concavity of the cylinder adapted to receive the container therein. The body may include a shell forming not more than half of a circumference of a cylinder, the container cavity being defined as a concavity of the cylinder adapted to receive the container therein.

The force aperture may be adapted to accept the container therethrough into the container cavity. The apparatus may define an insertion aperture distinct from the force aperture and adapted to accept the container therethrough into the container cavity. The force aperture may include a flexible membrane adapted to transmit the dispensing force therethrough to the container.

The apparatus may include a second sensor adapted to generate second sensor data. The second sensor may include a proximity sensor, a disposition sensor, a temperature sensor, a light sensor, an imager, a humidity sensor, an ultraviolet sensor, and/or an acoustic sensor. The second sensor may include a proximity sensor adapted to generate proximity data in response to an object proximity to the proximity sensor, and wherein the processor is adapted to determine whether the proximity data from the proximity sensor corresponds with a user handling the medication container, and in response to the proximity data corresponding with the user handling the medication container, activate the force sensor.

The apparatus may include a disposition sensor adapted to generate disposition data for the medication container. The processor may be adapted to activate the disposition sensor in response to the proximity data corresponding with the user handling the medication container, determine whether the force data from the force sensor and the disposition data from the disposition sensor in cooperation correspond with dispensing a medication from the medication container, and in response to the force data and the disposition data corresponding with dispensing the medication, register a medication event.

The apparatus may include a spine, the spine defining a spine cavity. The force sensor may be disposed in the spine cavity, and in communication with the container cavity. The apparatus may include a foot, the foot defining a foot cavity. The processor may be disposed in the foot cavity. The apparatus may include a shoe adapted to removably engage with the body, the shoe defining a shoe cavity.

The processor and the force sensor are removably engaged with the body. Some or all electronic components may be removably engaged with the body. Some or all non-mechanical components may be removably engaged with the body.

The body may be a label engaged with the container.

In another embodiment a method is provided that includes obstructing a dispensing force to a medication container from a first aspect, and passing a dispensing force to the medication container from a second aspect. The method includes generating force data from a transmitted force applied by the medication container in response to the dispensing force applied to the medication container, determining whether the force data corresponds with dispensing a medication from the medication container, and in response to the force data corresponding with dispensing the medication from the medication container, registering a medication event.

In another embodiment an apparatus is provided that includes means for obstructing a dispensing force to a medication container from a first aspect and means for passing a dispensing force to the medication container from a second aspect. The apparatus also includes means for generating force data from a transmitted force applied by the medication container in response to the dispensing force applied to the medication container, means for determining whether the force data corresponds with dispensing a medication from the medication container, and means for registering a medication event in response to the force data corresponding with dispensing the medication from the medication container.

BRIEF DESCRIPTION OF THE SEVERAL VIEWS OF THE DRAWINGS

Various objects, features, and characteristics will become more apparent to those skilled in the art from a study of the following Detailed Description in conjunction with the appended claims and drawings, all of which form a part of this specification. While the accompanying drawings include illustrations of various embodiments, the drawings are not intended to limit the claimed subject matter.

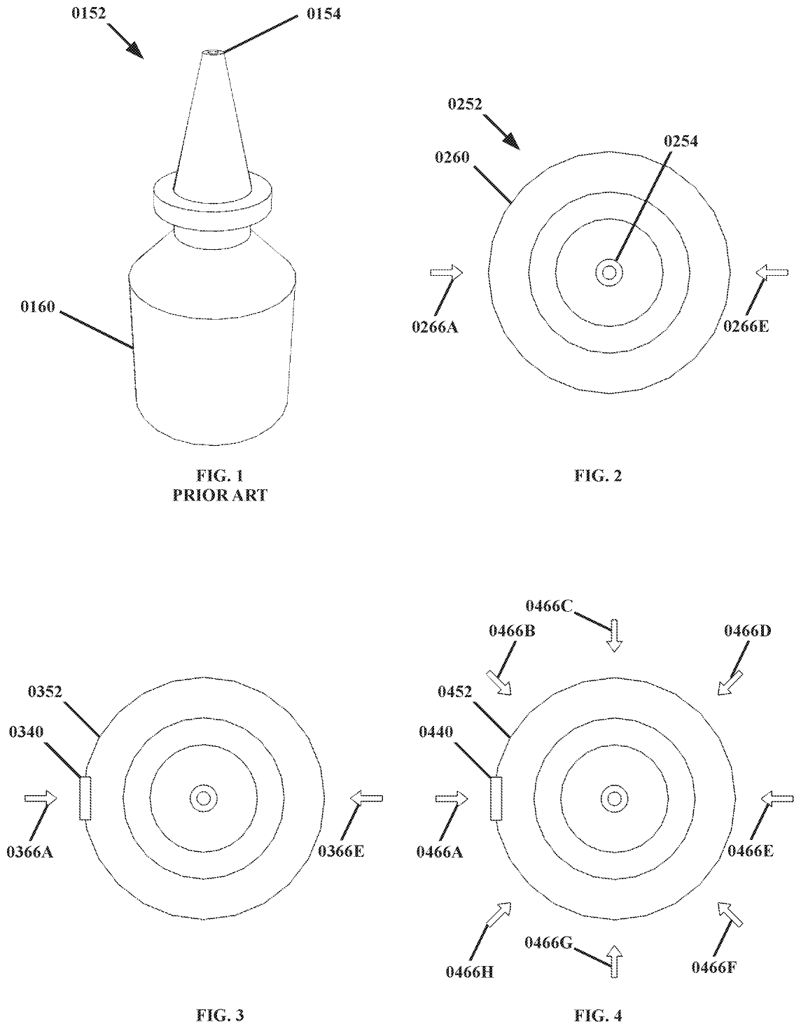

FIG. 1 depicts an example medication container, as known from the prior art.

FIG. 2 depicts an example arrangement of forces as may be applied to a medication container for dispensing medication, in top down view.

FIG. 3 depicts an example arrangement of forces as may be applied to a medication container for dispensing medication and a sensor as may detect such forces, in top down view.

FIG. 4 depicts another example arrangement of forces as may be applied to a medication container for dispensing medication and a sensor as may detect such forces, in top down view.

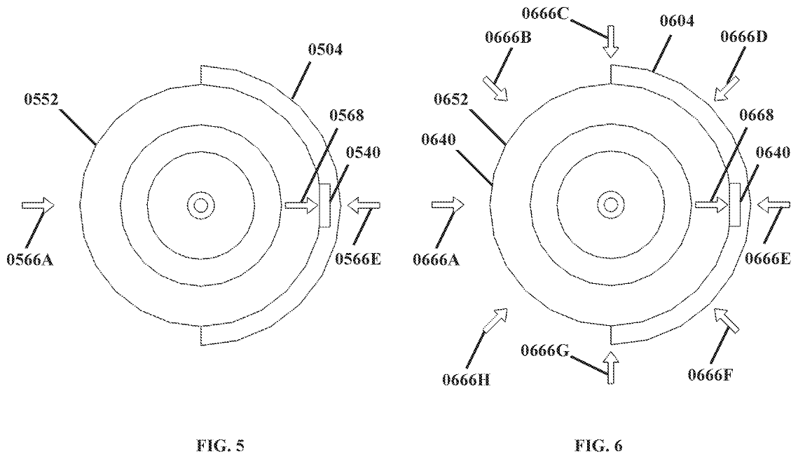

FIG. 5 depicts an example arrangement of forces as may be applied to a medication container for dispensing medication, a sensor as may detect such forces, and a body as may obstruct such forces, in top down view.

FIG. 6 depicts another example arrangement of forces as may be applied to a medication container for dispensing medication, a sensor as may detect such forces, and a body as may obstruct such forces, in top down view.

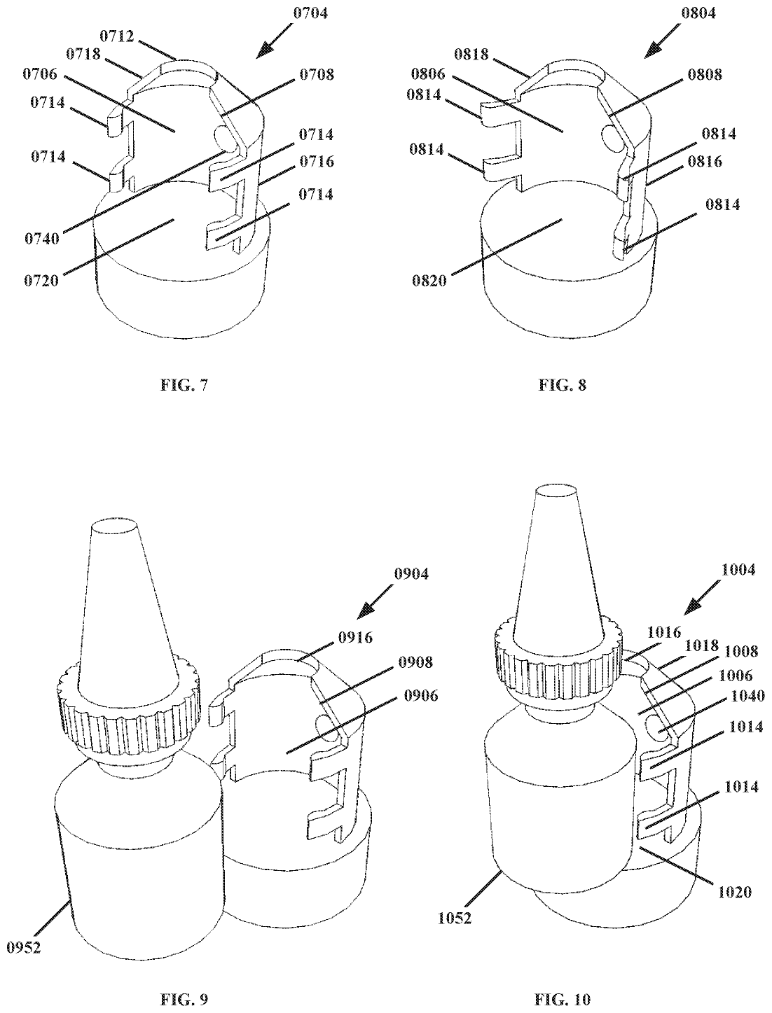

FIG. 7 depicts an example jacket for accommodating a container therein and indirectly determining the dispensing of contents therefrom, in perspective view.

FIG. 8 depicts another example jacket, showing restraints for the container spread open, in perspective view.

FIG. 9 through FIG. 12 show an example arrangement for inserting a container into a jacket, in perspective view.

FIG. 13 shows an example arrangement of a container disposed within a jacket with a container cap removed for dispensing medication, in perspective view.

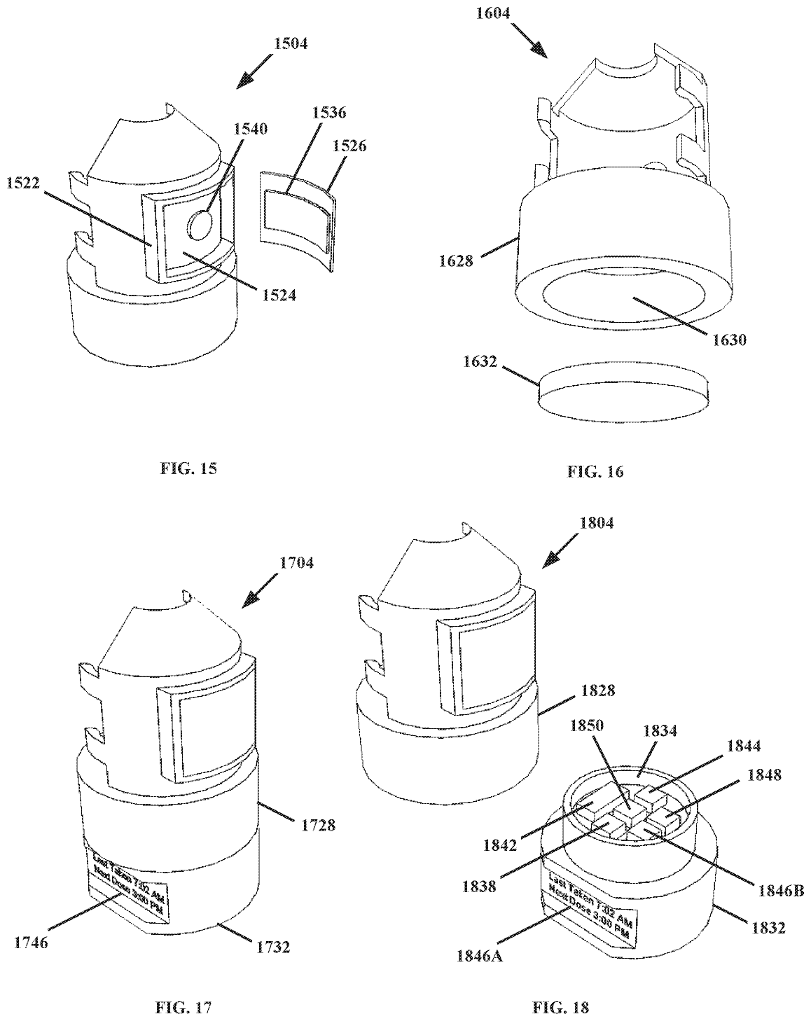

FIG. 14 and FIG. 15 show example spine component cavities for a jacket and components therein, in perspective view.

FIG. 16 through FIG. 18 show example foot component cavities for a jacket and components therein, in perspective view.

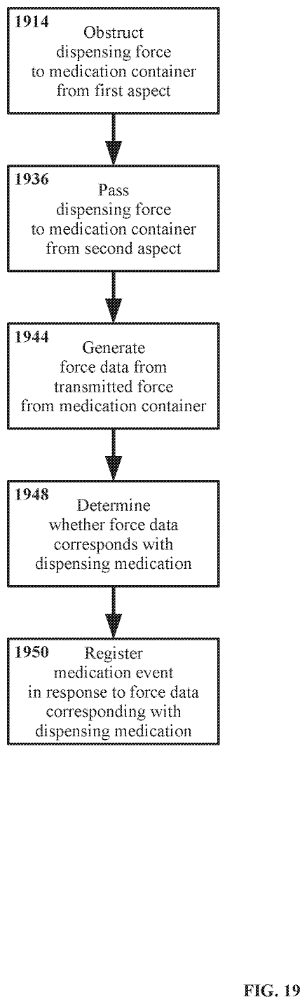

FIG. 19 depicts an example method for indirectly determining the dispensing of medication from a container, in flow chart form.

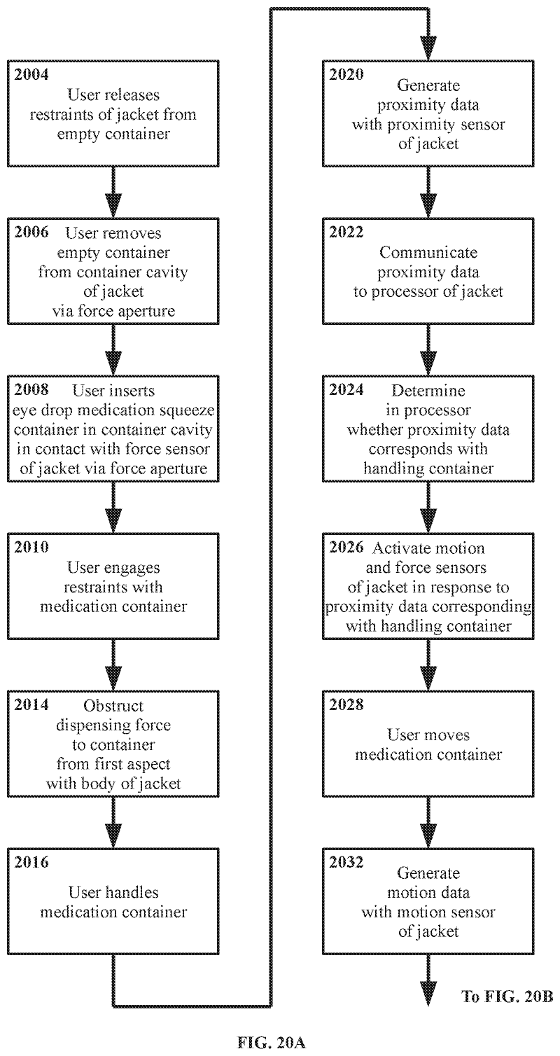

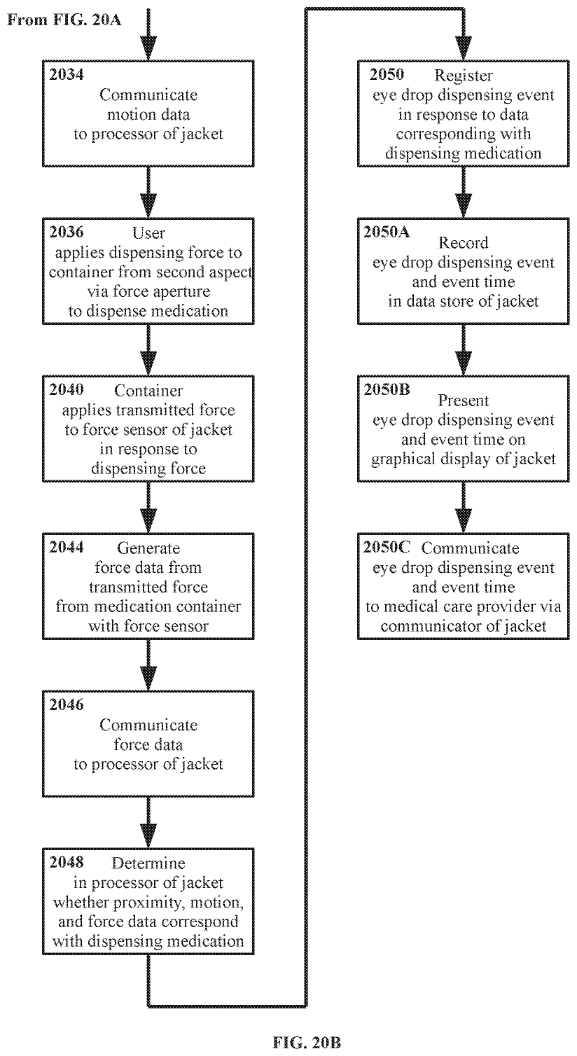

FIG. 20A and FIG. 20B depict another example method for indirectly determining the dispensing of medication from a container, in flow chart form.

FIG. 21 shows an example arrangement of circuit elements as may be utilized for indirectly determining the dispensing of medication from a container, in schematic form.

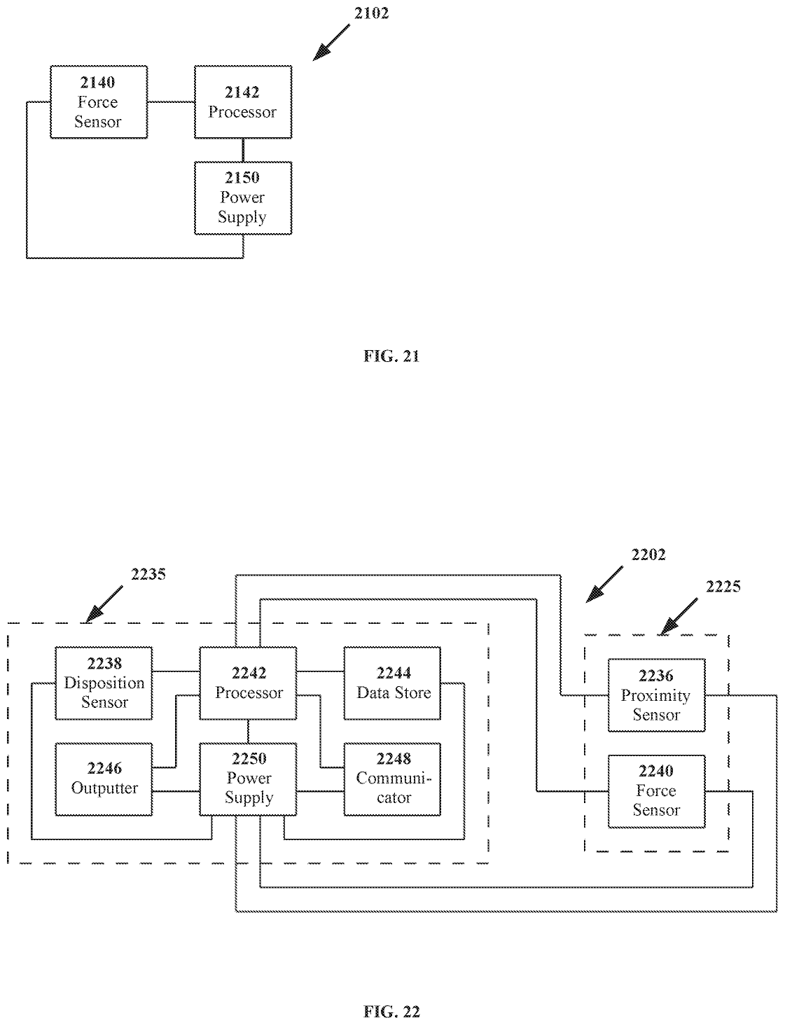

FIG. 22 shows another example arrangement of circuit elements as may be utilized for indirectly determining the dispensing of medication from a container, in schematic form.

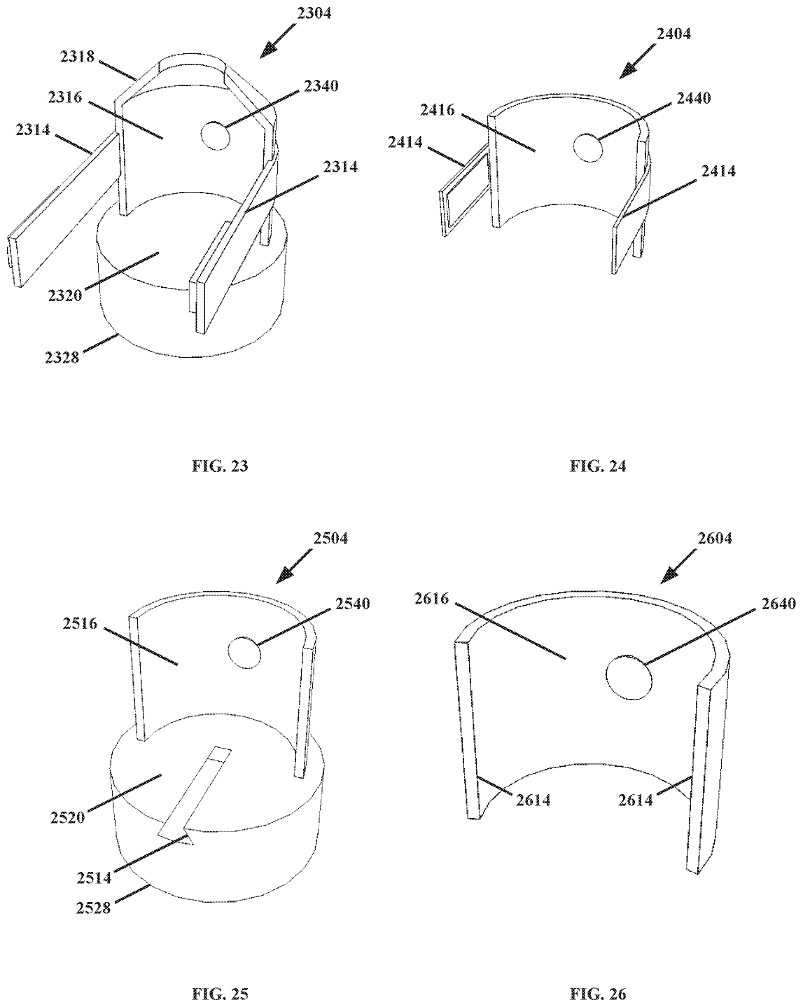

FIG. 23 through FIG. 26 show various example configurations for a body and/or restraints for indirectly determining the dispensing of medication from a container, in perspective view.

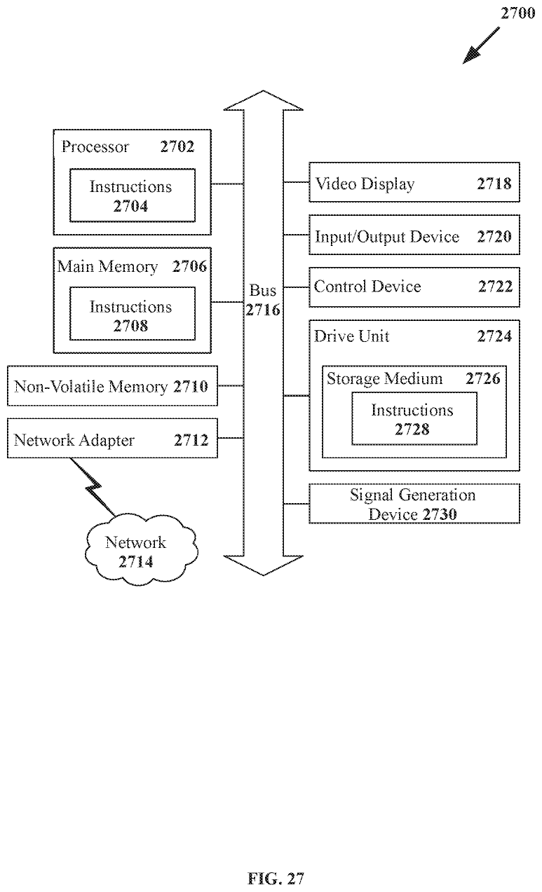

FIG. 27 is a block diagram illustrating an example of a processing system in which at least some operations described herein can be implemented.

The figures depict various embodiments described throughout the Detailed Description for the purposes of illustration only. While specific embodiments have been shown by way of example in the drawings and are described in detail below, the technology is amenable to various modifications and alternative forms. The intention is not to limit the technology to the particular embodiments described. Accordingly, the claimed subject matter is intended to cover all modifications, equivalents, and alternatives falling within the scope of the technology as defined by the appended claims.

### DETAILED DESCRIPTION OF THE INVENTION

Various embodiments are described herein that relate to indirectly determining the dispensing of medication from a container.

As an initial and non-limiting summary, the dispensing of medication from a container may be determined indirectly by detecting forces transmitted by the container when a user applies forces to that container, in order to dispense the medication. For example, medication may be dispensed from a squeeze bottle by applying a force to the side of the bottle (e.g., pressure with a finger). If a force sensor is disposed on the far side of the bottle from where force is applied, then the bottle may in turn press against that force sensor. The transmitted force—applied to the sensor by the container as force is applied to the container (e.g. by a user)—may be understood in at least some sense as being an “indirect force”. Thus, considering such indirect forces to determine whether medication has been dispensed similarly may be understood as “indirect sensing”.

By way of illustration, FIG. 1 shows an example arrangement of a squeeze bottle container 0152 in perspective view. As may be seen, the container 0152 includes a nozzle 0154 as may dispense liquid (e.g., eye drop medication) therefrom. As may be understood, applying inward force to the wall 0160 of the container 0152 decreases the interior volume of the container 0152, thus expelling the medication inside the container 0152 from the nozzle 0154.

FIG. 2 includes a similar container 0252 to that in FIG. 1, but from a top-down view. Again, the container 0252 may be seen to include a nozzle 0254 and a wall 0260. In addition, an arrangement of forces 0266A and 0266E as may be applied to the container 0252 are shown, for illustrative purposes. For example, forces 0266A and 0266E may be applied by a user (not shown) squeezing the container 0252 between his or her forefinger and thumb so as to dispense medication from the container 0252.

Now with reference to FIG. 3, a container 0352 again is shown (for the sake of simplicity the nozzle, wall, etc., of the container 0352 are not individually identified). In addition, a force sensor 0340 adapted to sense pressure applied thereto is shown disposed on the container 0352. When forces 0366A and 0366E are applied to the container 0352 as medication is dispensed, the force sensor 340 may generate force data in response to force 0366A (though not necessarily from force 0366E, which is applied at a different location and in a different direction).

However, it is noted that while FIG. 3 shows forces 0366A and 0366E that are aligned such that one such force (0366A) is directed to the force sensor 0340, forces applied from other directions not so aligned may not necessarily be directed to the force sensor 0340. The arrangement in FIG. 3 thus may not reliably provide force data as medication is dispensed from the container 0352.

For example, as shown in FIG. 4 several forces 0466A through 0466H are shown, as may be applied to a container 0452. Forces 0466A and 0466E may be detected by the force sensor 0440; however, other forces or pairs of forces, for example 0466C and 0466G, may not be so detected by the force sensor 0440. Given the arrangement shown, for direct forces applied to the container 0452, the force sensor 0440 as shown therein may only reliably detect a force such as 0466A that is aligned therewith. If forces may be applied directly to the container 0452 from other directions (e.g., forces 0466B through 0466H), then medication may be dispensed from the container 0452 without force data being generated by the force sensor 0440 in response thereto.

At least in principle it may be possible to arrange numerous force sensors so that any force as may be applied to dispense medication also may be detected by one such force sensor. However, in practice, such duplication of sensors may contribute to increased weight, cost, complexity power consumption, processor requirements (e.g., to monitor multiple sensors and evaluate multiple data streams therefrom), and so forth.

Turning to FIG. 5, an example arrangement of indirectly sensing forces associated with dispensing medication is shown. A container 0552 is shown, as may be similar to that shown previously in FIG. 2 through FIG. 4. However, a body 0504 is shown engaged with the container 0552, and extending across approximately half the circumference of the container 0552 as viewed from above. (The extent of the body 0504 as shown in FIG. 5 is an example only; bodies that extend across more or less than half the circumference of a container may be suitable.) A force sensor 0540 is disposed on the interior of the body 0504, proximate to the container 0552. (As illustrated in FIG. 5 the force sensor 0540 is physically in contact with the container 0552, however this is an example only and physical contact may not be required in all cases or at all times.)

Also, in FIG. 5, inward forces 0566A and 0566E are shown, as may be applied if a user were squeezing the container 0552 so as to dispense medication. However, the body 0504 obstructs the inward force 0556E from reaching the container 0552. For example, the body 0504 may be sufficiently rigid as to resist a reasonable level of force as may be applied by a user's fingers in dispensing medication, so that otherwise suitable applications of pressure (such as 0566E) may not be communicated to the container 0552 through the body 0504 so as to function as dispensing forces. In more colloquial language, with the body 0504 in the way squeezing may not dispense medication from the container 0552.

However, as may be seen inward force 0566A is not obstructed by the body 0552. Consequently, inward force 0566A may be sufficient to serve as a dispensing force and dispense medication from the container 0552. (It is noted that even though inward forces 0566A and 0566E may be applied together, e.g., by the thumb and finger of a user's hand, it is possible for one such force 0566E to be obstructed by the body 0504 while the other force 0566A is not obstructed.) In addition, the inward force 0566A applied to the container 0552 causes the container in turn to apply an outward force 0568 against the body 0504 and in particular against the force sensor 0540. That is, pushing against the container 0552 causes the container to push against the body 0504 and sensor 0540. The outward force 0568 may be detected by the force sensor 0540, and the force sensor 0540 may generate force data therefrom.

With regard to terminology, it is noted that although the terms “inward force” and “outward force” may be used with regard to FIG. 5, those terms are not necessarily descriptive for all possible embodiments. That is, the forces applied while dispensing a medication will not necessarily always be inward, nor will indirect forces resulting therefrom necessarily always be outward. It may be useful to consider more generally “applied force” for a force applied while dispensing medication, and “transmitted force” for a force that is secondary (e.g., transmitted from a container that is subject to an applied force). Thus, the forces 0566A and 0566E as shown may be considered applied forces, while the force 0568 as shown may be considered a transmitted force.

Furthermore, it may be useful to distinguish dispensing forces as distinct from applied forces more generally. Not all forces as may be applied to a container necessarily may be capable of dispensing medication or intended to dispense medication. Pressing the nozzle downward into a squeeze bottle at least arguably may be considered an applied force, but may not serve to dispense medication. Similarly, with a body 0504 in place as shown in FIG. 5, forces applied to the body 0504 may not necessarily be dispensing forces, in that forces applied to the body 0504 may not be communicated to the container 0552 in such way as to dispense medication. Thus, while force 0566A may be considered an applied force and/or a dispensing force, force 0566E may be considered an applied force but may not be considered a dispensing force (being obstructed from reaching the container 0552 by the body 0504).

Moving on to FIG. 6, another example arrangement of indirectly sensing forces associated with dispensing medication is shown. A container 0652 is shown, along with a body 0604 and a force sensor 0640, at least somewhat similar to what is shown in FIG. 5. In addition, FIG. 6 shows eight applied forces 0666A through 0666H in various directions with respect to the container 0652.

Typically, to facilitate a reliable determination that medication has been dispensed based on applied forces it may be preferable that all applied forces that result in medication being dispensed may be detected, and also that any applied forces that cannot result in medication being dispensed are in some manner excluded. In logical terms, in an ideal situation positive events should be detectable but false positives should not be detected.

Considering the arrangement in FIG. 6, applied forces 0666C through 0666G are obstructed by the body 0604. Because of obstruction by the body 0604, forces 0666C through 0666G may not cause material to be dispensed from the container 0652; forces 0666C through 0666G thus may be considered as false positives. In addition, forces 0666C through 0666G also may not produce a transmitted force 0668 such as is shown, since none of forces 0666C through 0666G may reach the container 0652 and also because none of forces 0666C through 0666G include a left-to-right component. Thus, at least certain false positive medication dispensing events may not be detected by the force sensor 0640.

Still with reference to FIG. 6 applied forces 0666A, 0666B, and 0666H are not obstructed by the body 0604, and thus may reach the container 0652; assuming the container 0652 is a squeeze bottle (as shown in the example of FIG. 6) applied forces 0666A, 0666B, and 0666H may serve as dispensing forces. In addition, as may be seen each of applied forces 0666A, 0666B, and 0666H is directed wholly or partially from left to right as illustrated; therefor, each of applied forces 0666A, 0666B, and 0666H may result in the container 0652 being pushed to the right, so that the container 0652 presses against the force sensor 0640 with transmitted force 0668 and the force sensor 0640 may generate force data therefrom. Thus, at least certain positive medication dispensing events may be detected by the force sensor 0640.

In sum with regard to FIG. 6, such an arrangement as shown therein may result in a transmitted force 0668 as may be detected by the force sensor 0640 when medication is dispensed, and may not result in a transmitted force 0668 as may be detected by the force sensor 0640 even when forces are applied thereto that otherwise may be suitable for dispensing medication if those forces are not oriented so as to actually dispense medication from the container 0652. Again, in logical terms, at least positive events may be detected but at least certain false positives may be excluded from detection.

In addition, several features of the arrangement in FIG. 6 (and as may be applicable to at least certain other embodiments as well) are to be noted.

The arrangement in FIG. 6 utilizes only a single pressure sensor 0640. While arrangements that may include more than one pressure sensor 0640 are not excluded, at least certain embodiments may function with only one pressure sensor 0640. The arrangement in FIG. 6 avoids the problem of detecting potential dispensing forces from many directions by using the body 0604 to restrict the application of dispensing forces to only such directions as may be detected by a single force sensor (those dispensing forces being indirectly sensed, in the form of transmitted forces applied via the container 0652).

Also, in the arrangement of FIG. 6 the force sensor 0640 may be considered to be internal; that is, either or both of the container 0652 and the body 0604 is disposed between the force sensor 0640 and the outside world. Thus, small and/or incidental forces may be blocked, absorbed, diminished, etc. by the container 0652 and/or the body 0604 before reaching the force sensor 0640. Likewise, the container 0652 and the body 0604 may provide at least some protection to the force sensor 0640 from environmental factors such as heat, cold, water, humidity, contamination, etc. Such an arrangement may at least contribute to protecting the force sensor 0640 from damage and/or malfunction, and/or may at least contribute to avoiding incidental data from being produced by the force sensor 0640 as may be misinterpreted as indicating that medication is being dispensed.

Turning now to FIG. 7, an example apparatus for indirect sensing of dispensing medication is shown. The apparatus includes a body 0704. As may be seen the example body 0704 includes a back obstacle 0716 that is an approximately semi-cylindrical half-shell, and a lower obstacle 0720 approximating a circular platform. The body 0704 also includes an upper obstacle 0718 in the form of a truncated conical shell. In addition, the body includes four restraints 0714. Certain functions and features of the body 0704 and elements thereof will be explained in greater detail subsequently. Broadly speaking, the body 0704 is adapted to accommodate a medication container, holding the container in place while still allowing medication to be dispensed therefrom.

The apparatus includes a force sensor 0740 disposed within the body 0704.

Several “negative space” elements also are shown in FIG. 7 with regard to the body. A container cavity 0706 is defined, adapted to accommodate the container therein. A force aperture 0708 also is defined, adapted to pass a dispensing force to the container (e.g., a compressive force for a squeeze bottle). In the example shown the force aperture 0708 also may serve as an insertion aperture adapted to enable a container to be inserted into the container cavity 0706 (and/or to be removed therefrom); thus for certain embodiments (though not necessarily all) it may be equivalent to refer to a single opening as either or both an insertion aperture and a force aperture. A dispensing aperture 0712 is also defined, adapted to enable medication to be dispensed from the container (e.g., by allowing a nozzle to extend therethrough). In some sense the container cavity 0706, force aperture 0708, and dispensing aperture 0712 may not be “things” per se; the container cavity 0706, force aperture 0708, and dispensing aperture 0712 are in common parlance spaces and holes, rather than physical structures. However, the container cavity 0706, force aperture 0708, and dispensing aperture 0712 are defined by physical structures that are present, e.g., the body 0704, and serve practical functions, and as such are identified and numbered herein for explanatory purposes.

The body 0704 as shown in FIG. 7 also includes four restraints 0714. In the example shown the restraints 0714 take the form of flexible and elastic arms or tabs; such restraints 0714 may serve to retain a container within the container cavity 0706, but may be released by being bent outward.

Turning to FIG. 8, such flexibility of restraints 0814 is illustrated for explanatory purposes. As may be seen the body 0804 in FIG. 8 is at least somewhat similar to that in FIG. 7, however in FIG. 8 the restraints 0814 are bent outward. In such configuration, a container may be inserted into or removed from the container cavity 0806 by moving the container laterally through the force aperture 0808 (while no longer being held in place by the restraints 0814). However, even with the restraints 0814 bent outward the body 0804 may retain a container within the container cavity 0806 other than via lateral motion through the force aperture 0808: the upper obstacle 0818 may obstruct a container from moving upward, the back obstacle 0816 may obstruct a container from moving backwards or sidewise, and the lower obstacle 0820 may obstruct a container from moving downward.

It is emphasized that the arrangement and operation of the restraints 0814 as shown in FIG. 8 is an example only. Other types of restraints, other numbers thereof, other manners of restraining a container, etc., also may be suitable. Moreover, while the restraints 0814 are shown to all be spread without a container present, in practice such restraints 0814 may be adapted to be pushed outward by the insertion and/or removal of a container itself, rather than requiring each restraint 0814 to be individually bent.

Now with reference to FIG. 9, a body 0904 at least somewhat similar to that in FIG. 8 is shown, along with a container 0952 as may be inserted into the container cavity 0906 of the body 0904 via the force aperture 0908. FIG. 10 through FIG. 12 may be understood similarly as a progression of a container being inserted into a body.

In FIG. 10, the container 1052 is shown to be approaching the restraints 1014 of the body 1004. The container 1052 is already partially over the lower obstacle 1020 and some portion of the container 1052 may be understood as at least partially within the container cavity 1006, however the container 1052 is not restrained by the restraints 1014, or fully obstructed by the upper obstacle 1018, and also is not engaged with the force sensor 1040.

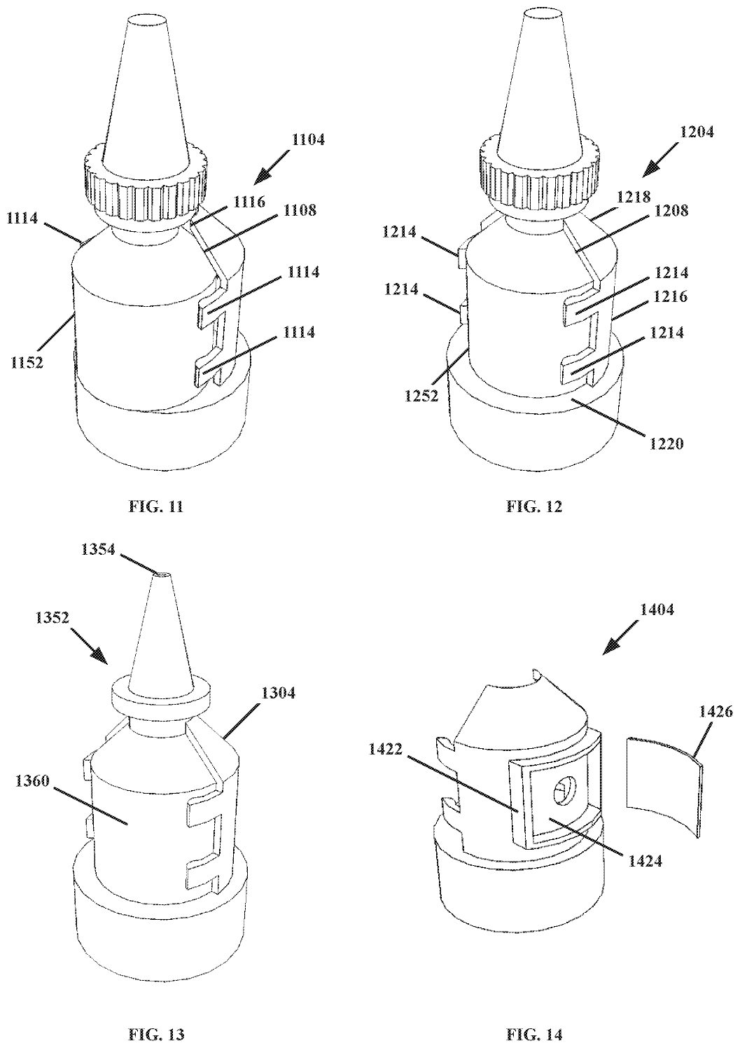

Turning to FIG. 11, as may be seen therein a container 1152 has been partially inserted into a body 1104 through the force aperture 1108 to a point such that the restraints 1114 (only three of which are visible in FIG. 11) are visibly spread apart to accommodate the container 1152. As also may be seen, the neck of the container 1152 is approaching (and may be partially within) the dispensing aperture 1116, and while the container cavity is not clearly visible (being occluded in FIG. 11 by the container 1152 itself) the container 1152 may be understood as being at least partly therein.

With reference to FIG. 12, a container 1252 is shown therein fully inserted into the container cavity (occluded by the container 1252 and so not individually identified) of a body 1204. The neck of the container 1252 may be seen to extend from the dispensing aperture (similarly not identified), and the restraints 1214 may be seen to be holding the container 1252 in place. The back obstacle 1216, upper obstacle 1218, and lower obstacle 1220 also are visible; as may be understood, motion of the container 1252 laterally out of the body 1204 except through the force aperture 1208 is obstructed by the back obstacle 1216, motion of the container 1252 upward out of the body 1204 is obstructed by the upper obstacle 1218, and motion of the container 1252 downward out of the body 1204 is obstructed by the lower obstacle 1220.

In FIG. 13, a container 1352 and body 1304 at least somewhat similar to those in FIG. 12 are shown, with the container 1352 similarly disposed within the body 1304. However, in FIG. 13 the container 1352 is shown without a cap, such that the nozzle 1354 of the container is exposed. As may be observed through a comparison of FIG. 12 and FIG. 13, a cap may be removed from a container 1352 without necessarily requiring the container 1352 to be removed from the body 1304, e.g., by unscrewing or popping off the cap. In the configuration shown in FIG. 13, the container may dispense medication from the container 1352 through the nozzle 1354, for example by applying force to the sidewall 1360 of the container 1352 (which remains exposed even with the container 1352 engaged with the body 1304 as shown). In practice a squeeze bottle container 1352 as illustrated in the example of FIG. 13 may be inclined or fully inverted for dispensing medication, e.g., to dispense an eye drop into a user's eye; the orientation in FIG. 13 is illustrative, and the orientation and configuration various embodiments of containers and/or bodies are not limited.

Now with reference to FIG. 14, as noted with regard to certain previous examples herein a body may include a force sensor therein. For example, in FIG. 7 the force sensor 0740 was shown as being on the surface of the body 0704 proximate and/or extending into the container cavity 0706 so as to receive forces transmitted via a container (not shown in FIG. 7) as may be disposed therein. However, while in certain embodiments it may be suitable to dispose a force sensor within a container cavity, other arrangements also may be suitable. For example, as shown in FIG. 14 the body 1404 is shown with a spine 1422 defining a spine cavity 1424 therein, and a spine cover 1426 as may be suitable for engaging with the spine 1422 so as to enclose the spine cavity 1424. As may be seen, a circular space (not individually numbered) is defined as part of the spine cavity 1424, as may accommodate a force sensor therein (e.g., in a configuration similar to that shown previously in FIG. 7). For example, a force sensor may be inserted from the spine cavity 1424 and extend into or at least proximate the container cavity (not directly visible in FIG. 14).

Turning to FIG. 15, a body 1504 again is shown with a spine 1522 defining a spine cavity 1524 therein, and a spine cover 1526 as may engage with the spine 1522 to enclose the spine cavity 1524. Where the example of FIG. 14 showed only a space as may accept a force sensor, in FIG. 15 a force sensor 1540 is visible within the spine cavity 1524. In addition, a proximity sensor 1536 (e.g., a capacitive sensor) also is shown engaged with the spine cover 1526; with the spine cover 1526 in place the proximity sensor 1536 would be within the spine cavity 1524 along with the force sensor 1540.

Now with reference to FIG. 16, a body 1604 is shown that includes a foot 1628, the foot 1628 in turn defining a foot cavity 1630 therein. A shoe 1632 also is shown; in the example of FIG. 16, the shoe 1632 serves as a cover for the foot cavity 1630, for example so as to enclose elements that may be disposed within the foot cavity 1630 (though the example arrangement of FIG. 16 does not illustrate any specific elements therein). The shoe 1632 as illustrated is a simple disk of material as may friction fit within the rim of the foot cavity 1630.

However, as may be seen in FIG. 17 a shoe (if present) may be more elaborate than a simple disk. In FIG. 17 a body 1704 is visible with a foot 1728 and a shoe 1732 engaged therewith. The shoe 1732 may be seen to include an outputter 1746 in the form of a graphical display. A graphical display 1746 such as is shown may present information relevant to a medication, a regimen, a patient, the apparatus itself, etc.; as illustrated the outputter 1746 presents a textual message: “Last Taken 7:02 AM Next Dose 3:00 PM,” though this is an example only and other information and/or forms of presentation (e.g., audio, video, telltale lights, etc.) may be suitable.

Turning to FIG. 18, another body 1804 is shown with a foot 1828, and a shoe 1832 also is shown therein. However, in the arrangement of FIG. 18 the shoe 1832 is shown disengaged from the foot 1828. As may be seen the shoe 1832 includes a graphical outputter 1846A similar to that in FIG. 17. However, in addition the shoe 1832 in FIG. 18 also defines a shoe cavity 1834 therein, with several elements disposed therein. In particular, the shoe cavity 1834 has disposed therein a disposition sensor 1838, processor 1842, data store 1844, audio outputter 1846B (as may perform a similar function as the graphical display 1846A of presenting information to the user, though being a separate element; other elements likewise may be duplicated, subdivided, etc. in various embodiments), communicator 1848, and power supply 1850. Thus, those and/or other elements may be disposed within the shoe 1832, and thus may be removably engaged with the body 1804.

Though not shown in FIG. 18, given the configuration of the shoe 1832 and the configuration shown in FIG. 17 it may be understood that the foot 1828 defines a foot cavity to accept the upper portion of the shoe 1832 therein. Thus, arguably the elements 1838, 1842, 1844, 1846B, 1848, and 1850 may be disposed in such a foot cavity when the shoe 1832 is engaged with the foot 1828; in such instance elements 1838, 1842, 1844, 1846B, 1848, and 1850 may be disposed within both the shoe cavity 1834 and the foot cavity (not shown in FIG. 18). Alternately, elements 1838, 1842, 1844, 1846B, 1848, and 1850 may be engaged directly within such a foot cavity without being in a shoe cavity 1834 (and/or without a shoe cavity even existing in such an embodiment).

Thus, as shown of FIG. 14 through FIG. 18 collectively various elements, including but not limited to sensors, processors, etc. may be disposed within various cavities defined within or on a given body. (More with regard to details and functions of such elements is described later herein.) Certain such cavities may be useful for at least some embodiments. For example, a spine cavity may facilitate convenient installation of a force sensor, proximity sensor, etc. during manufacturing (and indeed in some embodiments a spine cavity cover may be permanently engaged, e.g., glued in place). As another example, a shoe that includes electronics therein such as shown in FIG. 18 may be transferrable from one body to another. In such fashion bodies per se may be disposable, while certain significant elements (e.g., sensors, power supply, processor) may be reused by moving a shoe from one body to the next. In addition, it is noted that a shoe may be standardized to fit many different bodies, having different shapes and/or accommodating different medication containers. Similarly, a removable spine or spine cover likewise could have elements (such as a force sensor, proximity sensor, etc.) engaged therewith, thus also being transferrable from one body to another (including bodies of very different shape, size, etc., and suited for different medication containers). Thus, a body may—in at least certain embodiments—be a “dumb” device (possibly a disposable/recyclable device), such as 3D printed or injection molded plastic, with the “smarts” being disposed in reusable portions such as a spine cover, shoe, etc.

At this point it may be illuminating to describe certain advantages as may attach to various embodiments, in view of structure illustrated and described herein.

For example, in in configuring a body with a force sensor in a “half-shell” arrangement such as is shown in FIG. 7 (and elsewhere herein), the force sensor may sense pressure applied thereto by a medication container, rather than force applied to the medication container (or the body, etc.). This may be useful in avoiding certain “false positives,” at least in that the medication container itself may act as a sort of intermediary between forces in the outside world and the sensor. Thus, only a force that is sufficiently strong, lasting, etc. as to push the medication container against the force sensor may be registered by the force sensor. While squeezing a medication container to dispense eye drops (or gripping a medication container to remove a cap, shake out pills, etc.) may involve forces sufficient to be passed on by the container and registered by the force sensor, incidental bumps, touches, etc. may not. Thus, a body and medication container carried in a pocket, stored in a drawer, left on a counter, etc., may not register forces from ordinary handling, transport, and so forth.

Similarly, given such a half-shell arrangement the force sensor may be protected against certain forms of damage, such as rough handling. The medication container itself, in acting as an intermediary in transferring forces to the force sensor, may also be viewed as a sort of “shock absorber” protecting the force sensor. Force sensors may be prone to such damage by nature; attempting to make a force sensor insensitive to applied forces may be self-defeating, resulting in a sensor that cannot detect such forces. However, positioning a force sensor in such manner that certain incidental forces are restricted from reaching that sensor may be fruitful, without necessarily limiting the sensitivity of the sensor itself.

Further, such a half-shell configuration may (as already noted) restrict the direction(s) from which dispensing force may be applied to a medication container therein. Pressure applied from any direction except via the container insertion aperture (e.g., “the open side” of the body) may be blocked in part or in full by the body. If such forces are so blocked from reaching the container, then the container may not dispense medication in response. Consequently, only dispensing forces aligned with the container insertion aperture may result in medication being dispensed; given suitable placement of the force sensor, it may be arranged that all (or at least most) dispensing forces that can in fact dispense medication likewise are aligned with the force sensor, and are detectable thereby. Such an approach may avoid certain issues noted with regard to FIG. 2 through FIG. 6, related to detecting dispensing forces from various directions. Through the use of a half-shell arrangement as shown and described herein, a single force sensor may be sufficient to detect any force applied to a container as may actually cause medication to be dispensed.

Configuring a system so that all useful dispensing forces may be detected by a single sensor may provide certain benefits in itself. For example, use of multiple sensors typically may increase cost, weight, power draw, etc., and/or may present a greater “footprint” in terms of materials that are needed to manufacture a device and that must be accommodated when disposing of or recycling a device.

In addition, a half-shell configuration may facilitate visual and/or tactile guidance to a user as to where to apply dispensing forces. Rather than attempting (for example) to mark a container so as to indicate where to squeeze (e.g., so that a given sensor may detect the squeeze), leaving a gap such as the container insertion aperture through which the user may contact the container may provide sensory clues to the user. Configuring a half-shell structure may guide the user to naturally squeeze the medication container through the gap, possibly without the user having to consider the matter (and potentially without the user even being aware of being guided).

Furthermore, by leaving a gap a visual line of sight to the medication container may be maintained. Thus, labels identifying the medication, providing instructions for use, indicating use-by dates, etc. may remain visible even while the container is secured within the body and ready for use. In more colloquial terms, the medication is not being “covered up”; a user can see the container, the label, etc., and thus may distinguish one medication from another even while the medication containers are in respective bodies. In certain jurisdictions such visibility may even be legally required for at least some medications, or at least may be medically recommended. Likewise, optical scanners (e.g., reading bar codes, Quick Response (QR) codes, etc.) and/or other sensors such as radio-frequency identification (RFID) readers may be able to read containers/labels through the container insertion aperture even if the body proper is partly or entirely opaque to such sensors.

In addition, a half-shell arrangement adapted for lateral insertion of a container (as shown in certain examples herein) may have additional advantages with regard to accommodating a variety of containers. Containers may vary greatly in form, in part due to the needs of the medication, the intended manner for dispensing the medication, etc. A lateral-insertion half-shell configuration may be readily adapted for a wide variety of containers, where longitudinal insertion may not accommodate containers that are wider at the bottom or in the middle than at the top.

It is noted that at least certain embodiments of a half-shell body may be produced as integral objects, e.g., with no physical mechanisms such as push buttons, springs, latches, etc. Even for arrangements wherein a body may include two or more parts, such as a removable shoe, the body and/or the shoe may themselves be integral and without moving parts (e.g., using a friction fit, threads, etc. to engage). At least certain forms of restraints, such as those shown for example in FIG. 7, are also integral with the rest of the body, functioning as flexible tabs rather than having moving mechanical parts. (Although other arrangements also may be suitable.)

In addition, while the term “half-shell” may be used descriptively with regard to certain embodiments herein, it should not be understood as literal. For example, a given body is not required to cover half or even approximately half of a container or a circumference thereof; one-quarter or three-quarter coverage may be suitable for some embodiments, and coverage of a container is not limited. Nor is a body required to be or include a literal shell (e.g., thin, uniform, in direct contact with the container, etc.). Furthermore, while certain examples herein may show a half-shell configuration that accepts containers therein laterally and restricts vertical movement of containers therein, this too is an example and is not required for all embodiments.

Now with reference to FIG. 19, an example method for indirectly determining the dispensing of medication from a container is shown.

In the arrangement of FIG. 19, the application of dispensing forces to a medication container is obstructed 1914 from a first aspect. For example, as shown in certain previous figures the container may be disposed within a body (e.g., a “half-shell”), such that dispensing forces applied can only be applied to the container via the insertion aperture through which the container was inserted. In such instance, the first aspect under consideration may be directions other than through the insertion aperture. That is, applying force from the back or sides would be obstructed by the body, and thus the back and sides may be considered as being the first aspect. In other words, the first aspect is that direction (or those directions) from which force, if applied, is obstructed (e.g., by the body of an apparatus), so that such an applied force does not result in medication being dispensed from the container.

The manner by which force is obstructed 1914 from the first aspect is not limited. A body such as illustrated and described in certain previous examples herein may be suitable, but other arrangements also may be suitable. In addition, it is noted that force need not be fully prevented from reaching the container from the first aspect. For example, if sufficiently great force were applied to a body, presumably the body may deform or break; it is not required that a body be indestructible, impenetrable, etc. Rather, it may be sufficient merely to attenuate applied forces, or to block applied forces up to some maximum level, so that for example medication is not dispensed from the container in response to dispensing forces of typical magnitude from the first aspect. In such case, routine handling, storage, or even typical “squeezes” applied to (for example) an eye drop bottle from the first aspect would not cause medication to be dispensed, even if it were in theory possible to break or otherwise overwhelm the body with enough force.

It is noted that in referring to obstructing 1914 a dispensing force from the first aspect, it may not be required for a dispensing force to in fact be applied from the first aspect. That is, it may be sufficient that the effect of obstructing such forces is made manifest, for example, a container is engaged within a rigid body that would block a dispensing force from the first aspect if such a dispensing force were applied. Even if no actual dispensing force were ever applied to the container from the first aspect, it still may be reasonable to consider that such forces are blocked by the rigid body. While in a very strict sense it may be that what is being obstructed is a potential for dispensing forces to be applied, for clarity it is referred to herein that such forces are being obstructed. (Similarly, if motion of a container out of a container cavity is referred to herein as being obstructed, it should be understood that providing some means for carrying out that obstruction may be sufficient, whether or not any physical attempt ever is made to remove the container as obstructed.)

Moving on in FIG. 19, a dispensing force is passed 1936 to the medication via a second aspect. Referring again to examples such as illustrated in FIG. 13, the second aspect may correspond with the insertion opening/force opening shown therein to be defined in the body. Since in such an example the body does not extend to cover a medication container from all directions, a dispensing force applied to the container through the insertion opening/force opening would be passed to the container, e.g., would not be obstructed by the body. Thus for at least certain embodiments the second aspect might be described as “through the force aperture,” “in the direction a container would be inserted,” etc.

Again, the manner by which dispensing forces are passed along the second aspect is not limited. An opening such as a force aperture in a body may be suitable, but other arrangements also may be suitable. Likewise, dispensing forces applied along the second aspect need not be fully passed. For example, rather than being fully open (e.g., being literally an opening) a force aperture may be covered in part or in whole by flexible material, such as a layer of cloth or plastic film, etc. In such case the cloth or film may attenuate forces applied to the container, but forces still are passed sufficiently therethrough that medication may be dispensed from the medication container by applying a dispensing force from the second aspect. Such a “covered aperture” arrangement may still be considered to be (and may function as) a force aperture; even such a force aperture “isn't really a hole” in a colloquial sense, a flexible membrane may still pass forces to the container and thus still may be understood as being an aperture for forces (even if not being an aperture for the passage of physical matter therethrough; it may be that a covered force aperture may not be suitable to also serve as an insertion aperture).

Still with reference to FIG. 19, force data is generated 1944 from transmitted force that is applied by the medication container. That is, in response to a dispensing force being applied to the container (e.g., having been applied along the second aspect and passed 1936), the container then transmits that force, applying force in turn that is used (e.g., measured by a force sensor) to generate force data. It is noted that this step does not refer to generating force data directly from dispensing the force(s) applied to the container; rather, force data is generated from forces applied by the container. Although in some sense a force applied by a container may be related to a force applied to that container, in that if a container is pushed in the direction of a body and/or a sensor, the container will in turn push on the body/sensor. However, it is that indirect force—a force from the container—that is being used in step 1944 to generate force data.

A determination is made 1948 as to whether the force data corresponds with dispensing medication. For example, considering an eye drop bottle as an example the force applied thereto to dispense medication may exhibit certain ranges of magnitude, duration, rise and fall rates, overall curve shapes, etc. Such parameters may be sufficiently characteristic as to distinguish dispensing medication from dropping the container, an object bumping or pressing against the container in a pocket or bag, etc. In turn, the forces applied by the container in response to dispensing forces being applied thereto also may exhibit parameters that are sufficiently characteristic as to distinguish medication being dispensed from other events taking place. Consequently, the force data (which reflects the forces applied by the container) may likewise include sufficient information as to determine with at least some degree of confidence whether medication has been dispensed or not. For example, some standard for force data may be established, such as minimum and/or maximum force levels, curve shapes for plots of force over time, etc., and a comparison made between the force data and the force data standard. However, other arrangements also may be suitable.

As a particular example regarding force data, consider a distinction between dispensing medication from an eye drop bottle, squeezing the bottle with the cap off but with the bottle vertical so that only air is expelled therefrom, and squeezing the bottle with the cap in place. In dispensing medication, some resistance may be present when squeezing the bottle; liquid must be expelled, thus sufficient force must be applied to the container to overcome the stiffness of the container walls and the viscosity of the liquid medication, etc. In expelling only air, less resistance may be present (air being less viscous than certain liquids), and thus the overall force transmitted by the container, the rates of increase and decrease, etc. may be different Likewise, in expelling nothing (leaving the cap in place), the transmitted force again may be different, since the volume of the container may not be changing and nothing may be flowing from the nozzle thereof. Consequently, it may be possible to distinguish from the force data whether medication is in fact dispensed, or only air is expelled, or if the cap is left in place and nothing is expelled from the container. Thus, errors (e.g., forgetting to take the cap off) or deliberate deception (e.g., squeezing the bottle to mimic dispensing medication without actually dispensing any) may be identified based on evaluation of force data. These are examples only; such distinctions are not necessarily required to be made by all embodiments, nor are distinctions limited only to those presented as examples.

It is noted that the determination need not be absolute or perfect. For example, the determination may include the possibility of error, and false positives and/or false negatives may be acceptable in at least some embodiments. It may be preferable to avoid false positives and/or false negatives, but the determination in step 1948 (and likewise certain other determinations herein) are not limited only to perfect determination. Indeed, it may be suitable in certain embodiments to consider the degree of confidence, e.g., a determination may be recorded (for example as part of registration, below) along with a confidence level, e.g., “92% confidence that force data corresponds with dispensing medication,” “high confidence,” “class II confidence,” etc.

Continuing in FIG. 19, in response to the force data being determined 1948 to correspond with dispensing medication (e.g., based on the force data the determination is made that medication was dispensed), a medication event is registered 1950. The manner of registration is not limited; typical but non-limiting examples may include generating a record of the medication event (e.g. in a digital data store), displaying the event (e.g., on a graphical screen), and communicating the event to some third party such as an external database, a health care professional, etc. So long as the determination that medication has been dispensed is in some manner registered, the details thereof are not limited. In particular, it is noted that other information besides the medication event itself may be registered; for example, the time of the medication event may be registered. Other information including but not limited to the location of the medication event, the orientation/configuration of the container, the amount of medication dispensed, environmental conditions such as temperature or humidity, the force data itself (as distinct from determinations based thereon), etc. also may be registered.

Also, the registration 1950 of medication events does not necessarily exclude the registration of other events and/or data. For example, as noted with regard to step 1948 in certain embodiments it may be determined from force data whether certain faults have taken place with regard to dispensing medication, e.g., whether the cap was left on the container and nothing was dispensed, whether only air was dispensed because the container was not inclined, etc. While not necessarily considered medication events, such events nevertheless may be of interest. A pattern of only expelling air from the container may indicate difficulty in administering the drops, for example, or a pattern of leaving the cap on while squeezing the container may suggest absent-mindedness on the part of the user (a potentially illuminating symptom, medically) or deliberate deception (e.g., a clinical trial subject attempting to “game” the trial without taking the medication). In registering such events, even if no medication is dispensed, useful information may be obtained and/or useful conclusions drawn therefrom. Furthermore, it may be suitable to register the absence of medication being dispensed. That is, if medication were to be dispensed twice on a given day, and medication were only determined to be dispensed once or not at all, it may be useful to register that information. In such instance, a positive record may be created indicating that medication was not dispensed, rather than merely a lack of a record indicating that medication was dispensed.

Furthermore, if other sensors are present additional information not related to dispensing medication may be collected and/or registered. For example, considering a temperature-sensitive medication, if a temperature sensor is present on a container, in a jacket for the container, etc., it may be useful to register whether the temperature of that medication increases above a specified maximum (e.g., 25 degrees Centigrade). Maintaining medication at its proper temperature may or may not be considered to be part of compliance with a medication regimen, per se, however even if considered incidental and not part of compliance such information may be of use. For example, if a patient is not responding to a medication, and it is determined that the medication is not being kept below a specified temperature, this may present an opportunity for improving patient outcome by counseling the patient with regard to storage of their medication. Other sensors as may be suitable include, but are not limited to, a light sensor, an imager, a humidity sensor, an ultraviolet sensor, and an acoustic sensor.

Other data also may be similarly generated, evaluated, registered, etc.

Moving on to FIG. 20, another example method for indirectly determining the dispensing of medication from a container is shown. For purposes of clarity the example presented in FIG. 20 is relatively concrete, being specific to a jacket having particular properties and features, an eye medication container, the sensing of several types of data, etc. It is emphasized that the arrangement in FIG. 20 is an example only, and that not all embodiments necessarily will exhibit such features.

In the method of FIG. 20, the restraints of a jacket are released 2004 so that an empty container is freed from the jacket. The empty container is removed 2006 from the jacket via a force aperture (which given the removal and subsequent insertion of containers also may be considered as an insertion aperture and/or a removal aperture). It is noted that steps 2004 and 2006 may be performed together in certain embodiments. For example, considering a jacket such as is illustrated in FIG. 13, the restraints may disengage as the container is removed; lateral force is applied to the container so that the container “pops” free of the restraints (as may be considered by referring to FIG. 9 through FIG. 12 in reverse order). Thus, certain steps in a given embodiment of a method may be combined; likewise, certain steps may be combined, reordered, etc., so long as the overall functionality is achieved.

An eye drop medication squeeze container is inserted 2008 into the container cavity of the jacket via the force aperture, so as to be placed in contact with a force sensor on the jacket. The restraints are engaged 2010 with the medication container so as to retain the medication container in place. For example, the medication container may be obstructed from moving laterally out through the force aperture (or insertion aperture, etc.) by the restraints themselves, and while so held in the container cavity also may be obstructed from moving upward, downward, or laterally (other than through the force aperture) by upper, lower, and/or back obstacles. Such an arrangement is illustrated for example in FIG. 12, though other arrangements (including arrangements with no, fewer, or more obstacles, restraints, etc.) also may be suitable.

Again, as noted for certain embodiments steps 2008 and 2010 may be combined. Furthermore, for a medication container that may hold more than one dose of medication, steps 2004, 2006, 2008, and 2010 may be eliminated for most medication dispensing events (since the container will not need to be changed after each dose). Thus, in addition to combining/subdividing steps, steps also may be eliminated, and likewise also may be added, etc.

In addition, with regard to steps 2004, 2006, 2008, and 2010 it is considered for purposes of the arrangement in FIG. 20 that the user of the medication is performing those steps. Thus, the person using the eye drops is removing the empty bottle, inserting a fresh bottle, etc. Certain other steps below also refer to a user carrying out actions. This is presented for explanatory purposes, but is not limiting. Thus, the user may in fact insert a container into the jacket; however, other persons instead may insert the container, including but not limited to a caretaker, medical professional, manufacturer, etc. The distinction is made to distinguish for the sake of clarity that certain steps may be performed manually (e.g., inserting the container, squeezing the container to dispense medication) while other steps may be performed autonomously (e.g., a sensor generating force data). However, who (or what) performs which steps is not limited, and may vary from one embodiment to the next.

Continuing in FIG. 20, dispensing forces to the container are obstructed 2014 from a first aspect with the body of a jacket. For example, an arrangement such as shown in FIG. 12 may be utilized. (Though again, for such an arrangement the act of inserting 2008 the container into the jacket may arrange for such obstruction, without a distinct step being necessary.) A somewhat similar step was described previously with regard to FIG. 19.

The user handles 2016 the medication container. Since the medication container has been inserted 2008 into the jacket, handling 2016 the container may imply that the user is handling the jacket as well (and indeed for the example of FIG. 20, sensors are in the jacket for detecting such handling, as described hereafter).

A sensor of the jacket generates 2020 proximity data. For example, one or more capacitive sensors disposed on/in the jacket (e.g., in a spine thereof) may detect whether the container is being picked up, held, etc. through changes in capacitance, and may generate 2020 proximity data reflecting such changes in capacitance. The proximity data is communicated 2022 from the proximity sensor to a processor of the jacket (e.g., as may be disposed within a shoe cavity thereof). A determination 2024 is made in the processor as to whether the proximity data corresponds with a user handling the container (e.g., as in step 2016). As noted with regard to force data in step 1948 in FIG. 19, the proximity data may be sufficiently characteristic as to determine with at least some confidence whether the container is being handled, as opposed to whether the container is at rest, moving incidentally in a pocket or bag, etc. Also, as noted with regard to step 1948, the determination in step 2024 of FIG. 20 need not be perfect, and/or may include a level of confidence associated therewith.

Regardless of the manner of determination made in step 2024 and/or the confidence thereof, in response to a determination that the proximity data does correspond with the container being handled, motion sensors and force sensors in the jacket are activated 2026. For example, such activation may be performed by the processor, though other arrangements (e.g., a non-computational switching arrangement) also may be suitable. In the arrangement of FIG. 20, the motion sensors and force sensors typically may be powered off or otherwise inactive or minimally active; such low/absent activity may reduce power consumption, reduce computational requirements, reduce heat generation, etc. In such an arrangement, the determination in step 2024 may be understood as a sort of “gatekeeper” function: a positive determination 2024 may cause activation 2026 of additional sensors (e.g., as may provide further data relevant to whether medication is being dispensed), while a negative determination 2024 may leave those additional sensors quiescent.

As described, in the example arrangement of FIG. 20 proximity data from a proximity sensor is evaluated and (depending on the proximity data) two additional sensors may be activated, namely a motion/disposition sensor and a force sensor. Such an arrangement may be useful, for example in that a proximity sensor may require relatively little power to operate and so may be used as a “gate” in determining whether to activate additional sensors. However, it is emphasized that such an arrangement—one sensor activating another—is an example only, and is not required. Moreover, even when data from one sensor is considered in activating another sensor, embodiments are not limited only to a proximity sensor activating a disposition sensor and a force sensor. Any sensor(s) and/or any sensor data may be considered in activating any other sensor(s). For example, an embodiment with a force sensor and a motion sensor may use motion data to activate the force sensor (e.g., if the container's movement suggests that medication is being dispensed or is about to be), or an embodiment with a temperature sensor may use temperature data to activate a force sensor (e.g., if a refrigerated medication exhibits a rise in temperature as may indicate that the container has been removed from refrigeration so that medication may be dispensed). Other arrangements also may be suitable.

Still with reference to FIG. 20, the user moves 2028 the medication container. For example, a user may lift the container, tilt the container into an orientation suited for dispensing eyedrops into an eye, etc. (With regard to step 2016, “handling” the container refers to making physical contact with the container and/or jacket, or at least approaching close enough as to be detected by the proximity sensor. By comparison, moving 2028 the container refers to changing the position and/or orientation of the container and/or jacket engaged therewith. In practice moving 2028 the container also may include further handling the container, e.g., to move the container typically it may be necessary to touch the container. While collecting additional proximity data as the container is moved 2028 is not prohibited, neither is such additional proximity data collection required.)

Motion data is generated 2032 with a motion sensor of the jacket. For example, accelerometers, gyroscopes, etc. within the jacket may generate 2032 such motion data in response to the motion of the jacket (and the container therewith) in step 2028. The motion data is communicated 2034 to the processor of the jacket. It is noted that the term “motion data” is used for clarity; however, it should not be assumed that “motion data” necessarily is limited only to literal motion. For example, data indicating that “the container is inclined 85 degrees from vertical” may not, strictly speaking, refer to motion, but rather to orientation. Likewise, motion data as referred to herein may include position data, even if that position data does not literally refer to motion (e.g., change in position over time). Technically the orientation and/or position of a container may be referred to as “disposition,” however for purposes herein orientation and/or position (along with linear and angular acceleration, etc.) may be considered to be included in motion data unless otherwise noted.

The user applies 2036 dispensing force to the medication container via the second aspect (e.g., through a force aperture), so as to dispense medication. It is noted that the jacket passing the dispensing force is included herein in the example of FIG. 20; if the force is applied via the second aspect, that force will be passed without further action or intervention, for at least certain embodiments. Application of dispensing force via the second aspect as distinct from via the first was previously discussed with regard to FIG. 19.

The medication container applies 2040 a transmitted force to a force sensor of the jacket, in response to the dispensing force being applied 2036 to the container. For example, given a force sensor in line with the second aspect and disposed on or in a surface of the container cavity, squeezing the medication container to dispense medication may inherently cause the container to press against the force sensor. Continuing in FIG. 20, the force sensor generates 2044 force data from the transmitted force. The force data is communicated 2046 from the force sensor to the processor of the jacket.

The processor then determines 2048 whether the available combination of proximity data, motion data, and force data corresponds with medication being dispensed from the medication container. In the example of FIG. 20 all three types of data, gathered from three distinct sensors, are considered for the determination 2048. Such an arrangement may be useful, for example in providing higher confidence that medication has been dispensed, and/or for excluding instances wherein medication has not been dispensed. As a more concrete example, in dispensing medication from an eye drop squeeze container the container typically may be lifted, moved over the eye, and inclined in a characteristic sequence; the container may be handled throughout that time; and force may be applied in several pulses while the container is inclined (e.g., one pulse for each droplet). Any one such data set (proximity, motion, and force) may at least suggest that medication is dispensed, but if all three data sets exhibit characteristics of dispensing medication and exhibit those characteristics in proper timing (e.g., touch, lift, tilt, squeeze) a determination that medication has been dispensed may be made with considerable confidence.

However, not all embodiments necessarily will (or must) generate or consider multiple data sets. As noted previously, the example in FIG. 19 references only a single data set, force data. Nor are data sets limited only to those in the example of FIG. 20. For example, a cap sensor that generates data indicating whether the cap to a container remains in place (e.g., a pressure sensor, electrical contact pads, an optical sensor, etc.) may be suitable, as may a droplet sensor that generates data indicating whether medication physically exits the nozzle (e.g., an optical sensor, capacitance sensors, etc.), and other sensors and/or data sets also may be suitable for consideration.

Still with reference to FIG. 20, in response to the data (e.g., proximity data, motion data, and force data) having been determined 2048 to correspond with dispensing medication, an eye drop dispensing event is registered 2050. In particular, in the example of FIG. 20, registration includes at least three distinct elements 2050A, 2050B, and 2050C. That is, the eye drop dispensing event and the event time thereof are recorded 2050A in a data store of the jacket; the eye drop dispensing event and the event time thereof are presented 2050B on a graphical display of the jacket; and the eye drop dispensing event and the event time thereof are communicated 2050C to a medical care provider via a communicator of the jacket. Thus, the medication event and time thereof are registered redundantly, being recorded in the jacket, presented to the user, and forwarded to the user's physician (or other professional). As noted previously with regard to FIG. 19, other forms of and/or data in registration may be suitable, and embodiments are not limited with regard thereto. For example, the proximity, motion, and/or force data themselves may be registered, various non-medication events may be registered, etc.

It is emphasized that FIG. 19 and FIG. 20 represent example methods only, and should not be understood as limiting. In particular, other steps, other sensors and/or data, etc. may be equally suitable, and steps may be combined, subdivided, rearranged, etc. within the possibilities of function and logic.

Now with reference to FIG. 21 and FIG. 22 collectively, where certain previous illustrations have shown example physical arrangements, e.g., a jacket/body adapted to accept a container therein, FIG. 21 and FIG. 22 show arrangements for certain functional elements such as a processor, force sensor, etc.

Specifically, with regard to FIG. 21, an apparatus 2102 (e.g., in the form of a jacket for a container, though other arrangements may be suitable) is shown. The apparatus 2102 includes a force sensor 2140, a processor 2142 in communication with the force sensor 2140, and a power supply 2150 in communication with the force sensor 2140 and the processor 2142. Such an arrangement may correspond with certain other examples presented herein, such as the illustration of a jacket (and/or portions thereof such as a body and force sensor) in FIG. 7, and/or the method shown in FIG. 19. In both instances only a single sensor is shown and/or data thereof referred to, e.g., a force sensor 2104 as shown in FIG. 21. Reference to making determinations from data (which may for example be performed by a processor) also is made for example in FIG. 19. A power supply 2150 may be understood as implicit for at least such embodiments wherein elements such as sensors, a processor, etc. may be operated electrically.

Thus, the arrangement shown in FIG. 21 may in some sense be considered a “minimalist” configuration. (However, this is descriptive and not necessarily literal, and should not be taken to imply that every component shown in FIG. 21 necessarily must be present in all embodiments. For example, an embodiment that for example utilizes mechanical power applied by a user may not necessarily include a power supply per se.)

The force sensor 2140 is adapted to detect indirect force such as transmitted force from a container as a user applies forces to that container to dispense medication (or other contents), and to generate force data therefrom. The type and/or particular functionality of the force sensor is not limited. For example, piezoelectric force sensors as may generate varying electrical signals in response to forces being applied thereto may be suitable for a force sensor 2140, but this is an example only and other arrangements may be suitable. Similarly, certain force sensors may detect only that force above a minimum threshold is being applied, others may detect a continuum of forces and/or measure the level of force applied, etc.

The processor 2142 is adapted to process force data provided by the force sensor 2140, in particular to make a determination therefrom as to whether the force data corresponds with a user dispensing medication (and/or some other relevant and/or notable event). The processor 2142 also may be adapted to register a dispensing event in response to making a determination that force data does indeed correspond to medication being dispensed. In the example of FIG. 21, wherein no separate data store, etc. is shown to be present, registration may take the form of on-board data storage on the processor 2142 itself (though other arrangements may be suitable). Further, the processor 2142 may be adapted to monitor and/or control the force sensor 2140 and/or the power supply 2150. The type and operation of the processor 2142 are not limited. Typically, though not necessarily, electronic microprocessors adapted to execute instructions instantiated thereon may be suitable, but this is an example and is not limiting.

The power supply 2150 is adapted to provide power, e.g., electrical energy, to the force sensor 2140 and/or the processor 2142 as needed, such that the force sensor 2140 and/or the processor 2142 may carry out functions as described. The form of the power supply is not limited. Suitable power supplies may include but are not limited to batteries and wired current. However other power supplies, such as wireless power “harvesters,” piezoelectric or kinetic power supplies that generate electricity from manipulation of the apparatus 2102, etc. also may be suitable.

Turning to FIG. 22, another example apparatus 2202 is shown therein. As may be seen, the apparatus 2202 includes a force sensor 2240, a processor 2242, and a power supply 2250, as may be at least somewhat similar to elements described with regard to FIG. 21.

In addition, the apparatus 2202 includes a proximity sensor 2236 and a disposition sensor 2238 (as may also be referred to as a motion sensor). The proximity sensor 2236 and disposition sensor 2238 are in communication with the processor 2142 so as to provide data thereto (and/or to receive instructions therefrom, as noted with regard to the force sensor in FIG. 21), and also are in communication with the power supply 2250 so as to receive power therefrom (for embodiments wherein power is so required).

The proximity sensor 2236 is adapted to determine whether a user is proximate (e.g., physically contacting) the apparatus 2202 or some portion thereof, and to generate proximity data therefrom. The particulars of the proximity sensor 2236 are not limited. For example, certain proximity sensors may detect only physical contact, while others may detect that an object (e.g., the user) is near even without physical contact. Some proximity sensors may provide only binary data, e.g., the sensor is being touched or not, while others may provide information on how much area is being touched, with what pressure, etc. Suitable proximity sensors may include but are not limited to capacitance sensors as may determine proximity via changes in capacitance, pressure sensors as may determine proximity via pressures applied, conductivity sensors as may detect changes in conductivity (e.g., across the skin of a user contacting the apparatus 2202), etc., but other arrangements also may be suitable.

Similarly, the disposition sensor 2238 is adapted to determine one or more of the position, translation, orientation, rotation, etc. of the apparatus 2202 (and by extension a container engaged with the apparatus 2202). Again, the particulars of the disposition sensor 2238 are not limited. For example, certain disposition sensors may detect only active/current motion, e.g., translation and/or rotation that are currently taking place, while others may detect the position and/or orientation of the apparatus 2202 regardless of whether the apparatus 2202 is presently moving, and still others may detect linear and/or angular acceleration (rather than position or velocity per se), etc. Suitable disposition sensors may include but are not limited to accelerometers, gyroscopes, GPS and differential GPS sensors, etc.

As may be seen, the apparatus also includes a data store 2244, and outputter 2246, and a communicator 2248. Each of elements 2244, 2246, and 2248 may be adapted to perform functions relating to registration of medication events. For example, the data store 2244 may be adapted to register medication dispensing events by recording the event therein (and/or other information such as the dispensing time, sensor data, etc.). The outputter 2246 may be adapted to register dispensing events by presenting information to the user and/or other persons in the vicinity, for example by graphically displaying information, by audibly indicating that medication has been dispensed and/or is due to be dispensed, by telltales such as LEDs, etc. The communicator 2248 may be adapted to register dispensing events by sending information to some external party such as a database, a medical professional, a researcher, etc.

The above-described functions for elements 2244, 2246, and 2248 do not preclude those elements 2244, 2246, and 2248 from performing or being adapted to perform other functions (nor are additional functions prohibited for other elements). For example, the data store 2244 may store executable instructions thereon for instantiation onto the processor 2242, information on the medication, patient, etc., standards for carrying out various determinations described herein as being made, and so forth. The outputter may present information as may not be directly related to the taking of medication such as the current ambient temperature, the battery status of the apparatus 2202, etc. An outputter 2246 such as a graphical touch screen may accept inputs from users, thus serving as a control interface for the apparatus 2202 (though a separate interface also is not prohibited). A communicator 2248 may be adapted to receive software updates, submit requests for prescription refills, inform a manufacturer of a system malfunction, etc.

As may be seen, elements in FIG. 22 are grouped into a spine cluster 2225 and a shoe cluster 2235, as indicated by the dashed boxes. The spine cluster 2225 thus includes the proximity sensor 2236 and force sensor 2240, while the shoe cluster 2235 includes the disposition sensor 2238, processor 2242, data store 2244, outputter 2246, communicator 2248, and power supply 2250. As previously noted, certain elements may be disposed within different cavities (and/or other locations) on a given apparatus 2202, for example a spine cavity, a shoe cavity, etc. While such divisions of elements are examples only, and even when present may not have significant effect on the function of the elements themselves, divisions into spine and shoe clusters 2225 and 2235 may exist in practice (e.g., components may be in communication but still physically separated).

With regard to FIG. 23 through FIG. 26 collectively, certain previous examples have referred to jackets exhibiting mutual similarities, for example, utilizing flexure tabs as restraints. While this is done for explanatory purposes, in practice embodiments may vary widely in configuration, structure, etc. Certain examples of structural/functional variations, though by no means the only suitable examples, are presented in FIG. 23 through FIG. 26.

With reference now specifically to FIG. 23, therein a body 2304 is shown with a foot 2328, back obstacle 2316, upper obstacle 2318, and lower obstacle 2320, and a force sensor 2340 disposed therein, at least somewhat similar to certain previous examples (such as FIG. 7). In addition, the body 2304 includes restraints 2314 in the form of a two-part flexible band, e.g., of fabric, with hook-and-loop or similar thereon. Thus, a container may be inserted into the body, and secured in place by wrapping the restraints 2314 around the container and engaging the hook-and-loop. Where certain previous examples show flexure tabs as restraints, the nature of restrains as may be used in various embodiments is not limited. In addition to hook-and-loop straps, elastic bands, ties, etc. also may be suitable. Furthermore, restraints are not necessarily limited only to physical restraints; for a container that is constructed of or includes magnetic material (e.g., a container made with iron or steel, a label of magnetic material wrapped around a container, etc.) may utilize magnetic effects for restraint of the container within the body.

In FIG. 24, another example body 2404 is shown, with a back obstacle 2416 and a force sensor 2440 disposed therein. As may be seen, no dedicated upper or lower obstacle is present. However, the restraints 2414 are illustrated in the form of adhesive strips, such as may engage with a container disposed in the body 2404 (either fixedly or removably, for example depending on the choice of adhesive, the material of which the container is made, etc.). In being held in place by such adhesive restraints 2414, the container would be obstructed from moving either upward or downward out of the body, as well as laterally outward. Thus, in some sense the restraints 2414 may be considered to serve as upper and/or lower obstacles, in addition to serving as restraints. Other combinations and/or variations on the function of individual elements also may be suitable, and are not limited.

In addition, it is noted that the configuration shown in FIG. 24—a rigid semi-cylindrical back obstacle 2416 with a force sensor 2440 and adhesive restraints 2414—may approximate a label, may be fabricated and/or applied as a label, and/or may serve as a label. Thus, rather than inserting a container into the body 2404, it may be considered that a body 2404 is applied to a container. While in strict logical terms the two may be similar, in practice providing a body 2404 in the form of a semi-rigid label may differ for purposes of manufacturing and/or customer use. Manufacturing facilities may be already suited for mass-production of such labels and the application thereof to containers, for example, and in certain instances labels may be subject to fewer restrictions (e.g., with regard to certification of a medication delivery system) than distinct medical devices. Likewise, end users may already be accustomed to labels disposed on containers, and may be more amenable to using such a “half-shell label” than a larger and/or more obtrusive mechanism. Indeed, end users may not even be aware that such a body 2404 as shown in FIG. 24 is a functional apparatus (as opposed to being an inert label). Thus, such physical configurations and/or use of adhesive engagement may be advantageous for practical reasons.

In terms of function, it is noted that a body 2404 configured as a label may be constructed for example of rigid or semi-rigid material such as heavy paper, cardboard, various plastic films, etc. As described elsewhere herein it is not required that a given body completely prevent application of dispensing forces; thus, a label that is not entirely rigid and/or does not completely prevent deformation of a container therethrough may be suitable. In addition, it is noted that a body in the form of a label may be, in itself, a “dumb” device. That is, the label (e.g., body) may not have sensors, a processor, etc. disposed therein. In more colloquial terms, the label may be just a label, albeit a label with suitable extent, shape, and rigidity as to facilitate indirect sensing as described herein, etc. In such instance, smart components (sensors, processor, etc.) may be distinct from (though possibly engaged with) the body, e.g., being disposed within a foot, shoe, spine, etc. Such configuration may be suited for disposability on the part of the body (e.g., the body is thrown away with the container when the container is empty), while a foot, shoe, spine, etc. with sensors, processors, etc. therein are moved from an empty container to a full one and reused.

Even in instances wherein no active elements may be present on a body/label, such a “dumb” body/label is not necessarily required to be entirely inert; electrical traces for carrying electrical energy and/or other features may be present. For example, considering a label, electrical traces may be printed onto a label with conductive ink, applied as adhesive or laminated “press on” elements, etc. In such instances non-conductive material may also be used to insulate and/or physically protect those traces, e.g., nonconductive ink, laminated plastic film, sprayed-on lacquer, etc.

In addition, in certain embodiments sensors and/or other components may be part of a label. For example, an element such as a capacitive sensor for detecting proximity may be printed as layers of conductive ink with non-conductive material therebetween (e.g., nonconductive ink, laminated plastic film, sprayed-on lacquer, etc.) Alternately, an element such as a piezoelectric sensor for detecting forces may be provided in a relatively flat form and attached to a label as part of a fabrication process for labels, e.g., as a “press on” element in a multi-layer label (though at least in principle it may be possible to print a piezoelectric element and/or other elements directly, which also may be suitable). Thus, in at least certain embodiments, a body may take the form of a “smart label,” with some (though not necessarily all) “smart” functional elements therein.

Although such variations regarding a body that is “dumb,” “smart,” or somewhere in between (e.g., with functioning conductive paths but no other hardware) are described with regard to a label in this example, it is emphasized that such features apply similarly to other bodies regardless of form (e.g., bodies that are not labels), including but not limited to the body 2404 shown in FIG. 24.

Now with reference to FIG. 25, a body 2504 is shown with a back obstacle 2516 and a force sensor 2540 disposed therein, a lower obstacle 2520, and a foot 2528. As may be seen, a keyed groove is defined in the foot 2528. Such a keyed groove may accommodate a matching ridge in the bottom of a container as may be inserted into the body 2504. The keyed groove then may serve to secure the container in place, thus the groove may be considered as a restraint 2514. Ridges and grooves may also be configured so as to “twist lock,” e.g., slide in and then rotate, so that the container may not easily become disengaged laterally. In addition, the keyed groove also may function as an upper obstacle, since a container with a ridge matching the groove (wider at the bottom than the top) may not easily be removed upward; such an upper obstacle may not literally be “up” with respect to the container, but still may obstruct the container from moving up.

Such an arrangement as shown in FIG. 25 also may exhibit certain additional advantages. In practice, a medication monitoring system generally must be used properly in order to provide useful data. More bluntly, an instrument is of little use sitting on a shelf. Considering that patients failing to remember to take medication may be a factor in non-compliance, patients failing to remember to engage a medication container with a monitoring jacket also may be a possibility. However, a medication container with such a ridge may not easily be stood upright; with the container balanced on a narrow ridge, the container may be prone to tip over. Such a configuration may serve as a simple but readily visible prompt to engage a container with the body 2504 (e.g., so that the dispensing of medication may be detected and compliance determined therefrom). In more colloquial terms, seeing the bottle fall over may remind patients to make use of the smart jacket.

Turning to FIG. 26, another body 2604 is shown. The body 2604 includes a back obstacle 2616 and a force sensor 2640 disposed therein. The body 2604 also includes restraints 2614: as may be seen, the form of the body 2604 is slightly more than half of a cylinder. Consequently, the restraints 2614 may be considered as the entire edges of the body 2604, because those edges would extend far enough around a container (e.g., 210 degrees of circumference rather than 180 degrees) that the container could not easily fall out laterally. For a body 2604 as may be made of rigid but somewhat flexible material, the restraints 2614 in FIG. 26 may in some sense be understood as “full width” flexure tabs, similar in operation to those for example in FIG. 7 but extending across the entire width of the body 2604.

Alternately, certain medication containers, including but not limited to squeeze bottles, may themselves be at least somewhat flexible. Thus, even if the body 2604 as shown in FIG. 26 were completely rigid, a flexible container might be inserted and removed laterally by virtue of the flexibility of that container, while still being restrained for typical use and carriage via the material of the body 2604.

The arrangement in FIG. 26 does not illustrate explicitly numbered upper or lower obstacles, etc. Suitable obstacles are shown in other examples herein, and also may vary considerably. However, it is noted that upper and lower obstacles (and/or other functional elements) may in some sense be “virtually present”. That is, for a medication container that includes ridges near the top and bottom, if that container were inserted into the body 2604 illustrated the body 2604 itself may serve to obstruct upward or downward motion of the container due to the physical interference of the body 2604 with such ridges. Thus, what constitutes an upper obstacle, lower obstacle, back obstacle, etc. may to some extent be configured so as to cooperate with a particular container; even though the body 2604 may have no well-defined “thing” that may be pointed to as an upper obstacle, if the engagement between the body 2604 and the container is such that upward movement of the container out of the body 2604 is obstructed, then at least arguably a functional upper obstacle is indeed present. So long as the functions are carried out, the form and structure (or lack of structure) of upper obstacles, lower obstacles, etc. is not limited.

It is emphasized that the various configurations shown herein are examples only, and are not limiting. Other arrangements may be equally suitable. For example, while in certain examples herein bodies and/or other structures are shown as being opaque for clarity, it may be suitable for some embodiments to have a body, a portion thereof, etc. that is translucent or transparent. Such changes are not necessarily merely cosmetic considerations; a translucent body may facilitate the use of certain sensors, such as optical sensors; a transparent body similarly may facilitate certain sensors, and/or also may enable a label to be read through a body Likewise, while certain examples presented herein are shown as distinct from a container, e.g., in the form of a body that removably engages with the container, this also is not necessarily required for all embodiments. For example, in some embodiments a body may be injection molded in place around an existing container, e.g., by disposing the container in a mold and over-molding additional plastic (or other material) around the container. Such an overmolded body may be fixedly engaged with the container; thus, the restraints (insofar as distinct restraints may be identified) may be the overmolding geometry, and/or adhesion between the overmolded plastic and the plastic of the container. For example, if the container and overmolded body are both thermoplastics, the container and body may fuse together during overmolding. Such fusing may result in the container and overmolded body being essentially “one part,” e.g., fused to the point that removing the body may destroy the container; such permanent engagement is not required, but also is not prohibited.

Similarly, in certain embodiments the body may be fully integral with a container itself, for example being molded together with the container. In such instance, the “body” may essentially be an extension of the wall of the container, e.g., a thicker portion in the container wall that may serve to obstruct dispensing forces from a first aspect. (A force sensor for such an embodiment may be internal to the container wall/body, such as being molded inside the container wall. Alternately, a force sensor and/or other sensor may be attached to the container wall/body as or in a spine distinct from the container wall/body; likewise, a foot may be present and/or a separate shoe may be attached to the container, etc.) Such an integral arrangement may lack restraints in any meaningful sense; in colloquial terms the body may be just a thick spot in the wall. However, such an arrangement may be suitable for certain embodiments.

Now with reference to FIG. 27, therein is shown a block diagram illustrating an example of a processing system 2700 in which at least some operations described herein can be implemented. The processing system may include one or more central processing units (“processors”) 2702, main memory 2706, non-volatile memory 2710, network adapter 2712 (e.g., network interfaces), video display 2718, input/output devices 2720, control device 2722 (e.g., keyboard and pointing devices), drive unit 2724 including a storage medium 2726, and signal generation device 2730 that are communicatively connected to a bus 2716. The bus 2716 is illustrated as an abstraction that represents any one or more separate physical buses, point to point connections, or both connected by appropriate bridges, adapters, or controllers. The bus 2716, therefore, can include, for example, a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Engineers (IEEE) standard 1394 bus, also called “Firewire.”

In various embodiments, the processing system 2700 operates as a standalone device, although the processing system 2700 may be connected (e.g., wired or wirelessly) to other machines. In a networked deployment, the processing system 2700 may operate in the capacity of a server or a client machine in a client-server network environment, or as a peer machine in a peer-to-peer (or distributed) network environment.

The processing system 2700 may be a server, a personal computer (PC), a tablet computer, a laptop computer, a personal digital assistant (PDA), a mobile phone, a processor, a telephone, a web appliance, a network router, switch or bridge, a console, a hand-held console, a (hand-held) gaming device, a music player, any portable, mobile, hand-held device, or any machine capable of executing a set of instructions (sequential or otherwise) that specify actions to be taken by the processing system.

While the main memory 2706, non-volatile memory 2710, and storage medium 2726 (also called a “machine-readable medium) are shown to be a single medium, the term “machine-readable medium” and “storage medium” should be taken to include a single medium or multiple media (e.g., a centralized or distributed database, and/or associated caches and servers) that store one or more sets of instructions 2728. The term “machine-readable medium” and “storage medium” shall also be taken to include any medium that is capable of storing, encoding, or carrying a set of instructions for execution by the processing system and that cause the processing system to perform any one or more of the methodologies of the presently disclosed embodiments.

In general, the routines executed to implement the embodiments of the disclosure, may be implemented as part of an operating system or a specific application, component, program, object, module or sequence of instructions referred to as “computer programs.” The computer programs typically comprise one or more instructions (e.g., instructions 2704, 2708, 2728) set at various times in various memory and storage devices in a computer, and that, when read and executed by one or more processing units or processors 2702, cause the processing system 2700 to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Further examples of machine-readable storage media, machine-readable media, or computer-readable (storage) media include, but are not limited to, recordable type media such as volatile and non-volatile memory devices 2710, floppy and other removable disks, hard disk drives, optical disks (e.g., Compact Disk Read-Only Memory (CD ROMS), Digital Versatile Disks, (DVDs)), and transmission type media such as digital and analog communication links.

The network adapter 2712 enables the processing system 2700 to mediate data in a network 2714 with an entity that is external to the computing device 2700, through any known and/or convenient communications protocol supported by the processing system 2700 and the external entity. The network adapter 2712 can include one or more of a network adaptor card, a wireless network interface card, a router, an access point, a wireless router, a switch, a multilayer switch, a protocol converter, a gateway, a bridge, bridge router, a hub, a digital media receiver, and/or a repeater.

The network adapter 2712 can include a firewall that can, in some embodiments, govern and/or manage permission to access/proxy data in a computer network, and track varying levels of trust between different machines and/or applications. The firewall can be any number of modules having any combination of hardware and/or software components able to enforce a predetermined set of access rights between a particular set of machines and applications, machines and machines, and/or applications and applications, for example, to regulate the flow of traffic and resource sharing between these varying entities. The firewall may additionally manage and/or have access to an access control list which details permissions including for example, the access and operation rights of an object by an individual, a machine, and/or an application, and the circumstances under which the permission rights stand.

As indicated above, the computer-implemented systems introduced here can be implemented by hardware (e.g., programmable circuitry such as microprocessors), software, firmware, or a combination of such forms. For example, some computer-implemented systems may be embodied entirely in special-purpose hardwired (i.e., non-programmable) circuitry. Special-purpose circuitry can be in the form of, for example, application-specific integrated circuits (ASICs), programmable logic devices (PLDs), field-programmable gate arrays (FPGAs), etc.

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling others skilled in the relevant art to understand the claimed subject matter, the various embodiments, and the various modifications that are suited to the particular uses contemplated.

While embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Although the above Detailed Description describes certain embodiments and the best mode contemplated, no matter how detailed the above appears in text, the embodiments can be practiced in many ways. Details of the systems and methods may vary considerably in their implementation details, while still being encompassed by the specification. As noted above, particular terminology used when describing certain features or aspects of various embodiments should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless those terms are explicitly defined herein. Accordingly, the actual scope of the invention encompasses not only the disclosed embodiments, but also all equivalent ways of practicing or implementing the embodiments under the claims.

The language used in the specification has been principally selected for readability and instructional purposes, and it may not have been selected to delineate or circumscribe the inventive subject matter. It is therefore intended that the scope of the invention be limited not by this Detailed Description, but rather by any claims that issue on an application based hereon. Accordingly, the disclosure of various embodiments is intended to be illustrative, but not limiting, of the scope of the embodiments, which is set forth in the following claims.

What is claimed is:

1. An apparatus, comprising:
a body;
said body defining:
a container cavity adapted to accommodate a medication container such that said medication container moves with said body;
a force aperture adapted to pass a dispensing force to said container, said force aperture further being adapted to receive said medication container into said container cavity in an inward lateral motion;
a dispensing aperture adapted to enable a neck of said medication container to extend therethrough so as to enable said container to dispense medication while said container is in said container cavity;
said body comprising:
a plurality of flexible arms adapted to releasably restrain said container within said container cavity;
a back obstacle adapted to obstruct an outward lateral motion of said container from said container cavity other than via said force aperture;
an upper obstacle adapted to obstruct an upward vertical motion of said container from said container cavity, said dispensing aperture being defined therein;
a lower obstacle adapted to obstruct a downward vertical motion of said container from said container cavity;
a spine engaged with said body, said spine defining a spine component cavity therein such that when said spine is engaged with said body said container cavity is between said force aperture and said spine component cavity;
a foot engaged with said body, said foot defining a foot component cavity;
a proximity sensor disposed within said spine component cavity and adapted to generate proximity data in response to a proximity of a finger thereto from outside the body;
a disposition sensor disposed within said foot component cavity and adapted to generate disposition data comprising at least one of position data, translation data, orientation data, and rotation data in response to a disposition of said body comprising at least one of a position, translation, orientation, and rotation of said body;
a force sensor disposed within said spine component cavity and adapted to generate force data in response to a transmitted force applied thereto from said medication container;
a processor disposed within said body, in communication with said force sensor, said proximity sensor, and said disposition sensor;
a data store in communication with said processor;
an outputter in communication with said processor;
a communicator in communication with said processor;
wherein:
said processor is adapted to:
determine whether said proximity data from said proximity sensor corresponds with a dispensing proximity of said finger to said proximity sensor associated with dispensing said medication from said container;
in response to said proximity data corresponding with said dispensing proximity, activate said disposition sensor and said force sensor;
determine whether said disposition data from said disposition sensor corresponds with a dispensing disposition of said body with said container therein associated with dispensing said medication from said container;
determine whether said force data from said force sensor corresponds with a dispensing force being applied to said medication container and said transmitted force being applied to said force sensor by said medication container in response to said dispensing force;
in response to said disposition data corresponding with said dispensing disposition and said force data corresponding with said dispensing force, register a medication event;
said body, said force aperture, and said force sensor are configured such that said transmitted force is generated in response to said dispensing force being applied to said medication container through said force aperture, and such that said transmitted force is not generated in response to said dispensing force being applied to said medication container other than through said force aperture;
registering said medication event comprises:
storing said medication event and a medication event time thereof in said data store;
outputting said medication event and said medication event time via said outputter; and
communicating said medication event and said medication event time to an external entity via said communicator.

2. An apparatus, comprising:
a body;
said body defining a container cavity adapted to accommodate a medication container such that said medication container moves with said body, and a force aperture adapted to pass a dispensing force to said container, said force aperture further being adapted to receive said medication container into said container cavity;
said body comprising a restraint adapted to releasably restrain said medication container in said container cavity;
a force sensor adapted to generate force data in response to a transmitted force applied thereto from said medication container; and
a processor in communication with said force sensor and adapted to:
determine whether said force data from said force sensor corresponds with dispensing a medication from said medication container;
in response to said force data corresponding with dispensing said medication, register a medication event;
wherein:
said body, said force aperture, and said force sensor are configured such that said transmitted force is generated in response to said dispensing force being applied to said medication container through said force aperture, and such that said transmitted force is not generated in response to said dispensing force being applied to said medication container other than through said force aperture;
registering said medication event comprises at least one of:
storing said medication event and a medication event time thereof;
outputting said medication event and said medication event time; and
communicating said medication event and said medication event time to an external entity.

3. The apparatus of claim 2, wherein:
said restraint comprises at least one of:
a flexible arm adapted to releasably restrain said container within said container cavity via mechanical interference therewith;
an adhesive strip adapted to restrain said container within said container cavity via adhesion thereto;
a two-part hook-and-loop band adapted to restrain said container within said container cavity via hook-and-loop engagement of said band around said container;
an elastic band adapted to restrain said container within said container cavity via said elastic band being disposed around said container;
a tie cord adapted to restrain said container within said container cavity via said tie cord being tied around said container;
a magnet adapted to restrain said container within said container cavity via magnetic engagement between said magnet and said container.

4. The apparatus of claim 2, wherein:
said body comprises a shell forming at least half of a circumference of a cylinder, said container cavity being defined as a concavity of said cylinder adapted to receive said container therein.

5. The apparatus of claim 2, wherein:
said force aperture is adapted to accept said container therethrough into said container cavity.

6. The apparatus of claim 2, comprising:
an insertion aperture distinct from said force aperture and adapted to accept said container therethrough into said container cavity.

7. The apparatus of claim 2, wherein:
said force aperture comprises a flexible membrane adapted to transmit said dispensing force therethrough to said container.

8. The apparatus of claim 2, comprising:
a second sensor adapted to generate second sensor data.

9. The apparatus of claim 8, wherein:
said second sensor comprises at least one of a proximity sensor, a disposition sensor, a temperature sensor, a light sensor, an imager, a humidity sensor, an ultraviolet sensor, and an acoustic sensor.

10. The apparatus of claim 8, wherein:
said second sensor comprises a proximity sensor adapted to generate proximity data in response to an object proximity to said proximity sensor;
wherein said processor is adapted to:
determine whether said proximity data from said proximity sensor corresponds with a user handling said medication container;
in response to said proximity data corresponding with said user handling said medication container, activate said force sensor.

11. The apparatus of claim 10, comprising:
a disposition sensor adapted to generate disposition data for said medication container;
wherein said processor is adapted to:
activate said disposition sensor in response to said proximity data corresponding with said user handling said medication container;
determine whether said force data from said force sensor and said disposition data from said disposition sensor in cooperation correspond with dispensing a medication from said medication container; and
in response to said force data and said disposition data corresponding with dispensing said medication, register a medication event.

12. The apparatus of claim 2, comprising:
a spine, said spine defining a spine cavity.

13. The apparatus of claim 12, wherein:
said force sensor is disposed in said spine cavity, and in communication with said container cavity.

14. The apparatus of clam 2, comprising:
a foot, said foot defining a foot cavity therein.

15. The apparatus of claim 14, wherein:
said processor is disposed in said foot cavity.

16. The apparatus of claim 2, comprising:
a shoe adapted to removably engage with said body, said shoe defining a shoe cavity therein.

17. The apparatus of claim 2, wherein:
said processor and said force sensor are removably engaged with said body.

18. The apparatus of claim 2, wherein:
said body comprises a label engaged with said container.

19. A method, comprising:
obstructing a dispensing force to a medication container from a first aspect;
passing a dispensing force to said medication container from a second aspect;
generating force data from a transmitted force applied by said medication container in response to said dispensing force applied to said medication container;
determining whether said force data corresponds with dispensing a medication from said medication container; and
in response to said force data corresponding with dispensing said medication from said medication container, registering a medication event.

20. An apparatus, comprising:
means for obstructing a dispensing force to a medication container from a first aspect;
means for passing a dispensing force to said medication container from a second aspect;
means for generating force data from a transmitted force applied by said medication container in response to said dispensing force applied to said medication container;
means for determining whether said force data corresponds with dispensing a medication from said medication container; and
means for registering a medication event in response to said force data corresponding with dispensing said medication from said medication container.
